SEQUENCE LISTING (13 sequences)

SEQ ID NO: 1 - DNA, 6876 bp, Homo sapiens
agacacgtgctggggcgggcaggcgagcgcctcagtctggtcgcctggcggtgcctccggccccaacgcgcccgggccgccgcgggccgcgcgcgccgatgcccggctgagtcactggcagggcagcgcgcgtgtgggaaggggcggagggagtgcggccggcgggcgggcggggcgctgggctcagcccggccgcaggtgacccggaggccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtacttggtggaaagaacctctcaacattgtcagttttctattttgctttgccagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaa

SEQ ID NO: 2 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 3 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 4 - protein, 764 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFIRHVNIALIQR

SEQ ID NO: 5 - protein, 158 aa, Homo sapiens
AVKGEVLKVGNKSCENIHLHSEAVLCTVPNDLLKLNSELNIEWKQAISSTVLGKVIVQPDQNFTGLIAGVVSISTALLLLLGFFLWLKKRKQIKDQFPNSSQNGSCRQVQYPLTDMSPILTSGDSDISSPLLQNTVHIDLSALNPELVQAVQHVVIGP

SEQ ID NO: 6 - protein, 190 aa, Homo sapiens
MKSKSKSLAECFPYDKPLIMKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQ

SEQ ID NO: 7 - protein, 214 aa, Homo sapiens
AVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 8 - DNA, 154 bp, Artificial Sequence
  Synthetic
  misc_feature (134)..(135): n is a, c, g, or t
  misc_feature (138)..(138): n is a, c, g, or t
  misc_feature (140)..(140): n is a, c, g, or t
gtttgtccacagagacttggctgcaagaaactgtatgggaagatggcggatctggaggagcagttgtctgatgaagagaagaatccaactgtaaaagatcttattggctttggtcttcaagtagccaaaggcannaantntctgcaagcaaaaa

SEQ ID NO: 9 - DNA, 262 bp, Artificial Sequence
  Synthetic
ttgtctgatgagagaagtggtcctttggcgtgctcctctgggagctgatgacaagaggagccccaccttatcctgatgtaaacacctttgatataactgtttacttgttgcaagggagaagactcctacaacccgaatactgcccagaccccttatatgaagtaatgctaaaatgctggcaccctaaagccgaaatgcgcccatccttttctgatgtttgtcgccagaaggaagatggcggatctggaggagcagttgtctg

SEQ ID NO: 10 - DNA, 169 bp, Artificial Sequence
  Synthetic
aagagtttagcagaatgcttcccatatgataaacctctcataatgaaggcccccgctgtgcttgcacctggcatcctcgtgctcctgtttaccttggtgcagaggagcaatggggagtgtaagcctcccaagtagctgagactacagggtggtgatgaagagtaaatca

SEQ ID NO: 11 - DNA, 90 bp, Artificial Sequence
  Synthetic
  misc_feature (41)..(41): n is a, c, g, or t
  misc_feature (43)..(43): n is a, c, g, or t
cttctccacggttcctgggcaccgaaagattgactgaagangngatgcaaaccagaagggacacacagttttgacggcatttggaaggcc

SEQ ID NO: 12 - DNA, 335 bp, Artificial Sequence
  Synthetic
tgtgtgcattccctatcaaatatgtcaacgacttcttcaacaagatcgtcaacaaaaacaatgtgagatgtctccagcatttttacggacccaatcatgagcactgctttaataggattgactttgaagatgtgattgcagaaccagaagggacacacagttttgacggcatttggaaggccagcttcaccaccttcactgtgacgaaatactggttttaccgcttgctgtctgccctctttggcatcccgatggcactcatctggggcatttacttcgccaattgttcgcacaaagcaagccagattctgccgaaccaatggatcgatctgcca

SEQ ID NO: 13 - DNA, 201 bp, Artificial Sequence
  Synthetic
tgcatttgcacagtataacttggaccagtttactccagtaaaaattgaaggttatgaagatcaggcatgtcaacatcgctctaattcagagataatctgttgtaccactccttccctgcaacagctgaatctgcaactccccttcaatgatgttcggttactgcttaataatgacaatcttctcagggaaggagcagccca

(19) United States
(12) Patent Application Publication
(10) Pub. No.: US 2022/0252603 A1
(43) Pub. Date: Aug. 11, 2022

(54) METHOD FOR TREATING CANCER PATIENTS USING C-MET INHIBITOR

(71) Applicant: APOLLOMICS INC. (HANGZHOU), Hangzhou, Zhejiang (CN)

(72) Inventors: Lan YANG, Hangzhou, Zhejiang (CN); Qian SHI, Hangzhou, Zhejiang (CN); Sanjeev REDKAR, San Ramon, CA (US); Guoliang YU, Incline Village, NV (US); Fabio Maurizio BENEDETTI, Redwood City, CA (US); Xiaoling ZHANG, Foster City, CA (US); Biao MA, Hangzhou, Zhejiang (CN)

(21) Appl. No.: 17/616,203

(22) PCT Filed: Jun. 8, 2020
(86) PCT No.: PCT/CN2020/094824

§ 371 Date: Dec. 3, 2021

(30) Foreign Application Priority Data
Jun. 6, 2019 (CN) PCT/CN2019/090294
Jun. 25, 2019 (CN) PCT/CN2019/092706
Oct. 8, 2019 (CN) PCT/CN2019/109906

(51) Int. Cl. G01N 33/574

(52) U.S. Cl. CPC G01N 33/57407

(57) ABSTRACT

Provided herein is related generally to the field of molecular biology and growth factor regulation. More specifically, provided herein are methods useful for treating cancer patient using c-Met inhibitor based on the identification of an increased c-Met expression and at least one c-Met gene alteration, e.g. c-Met mutation, c-Met fusion gene and c-Met gene amplification.

### CROSS-REFERENCE TO RELATED APPLICATIONS

This application claims priority to PCT/CN2019/090294, filed Jun. 6, 2019, PCT/CN2019/092706, filed Jun. 25, 2019, and PCT/CN2019/109906, filed Oct. 08, 2019, the disclosure of which is incorporated herein by reference.

### FIELD OF THE INVENTION

The present invention generally relates to cancer treatment. In particular, the present invention relates to methods for treating cancer patients using c-Met inhibitor based on c-Met gene alteration, e.g., c-Met gene mutation, c-Met fusion gene, c-Met gene amplification or c-Met expression level.

### BACKGROUND

The Hepatocyte Growth Factor Receptor, also named as c-Met, is a receptor tyrosine kinase that regulates a wide range of different cellular signaling pathways, including those involved in proliferation, motility, migration and invasion. Due to its pleotropic role in cellular processes important in oncogenesis and cancer progression, c-Met has been shown to be over-expressed in a variety of malignancies, such as Small Cell Lung Cancer (SCLC) and NSCLC (Olivero et al., Br J Cancer, 74: 1862-8 (1996) and Ichimura et al., Jpn J Cancer Res, 87:1063-9 (1996)) and considered as an important target in anticancer therapy.

Inhibitors specifically against c-Met represent an attractive novel targeted therapeutic approach. For example, the effectiveness of a novel small molecule specific inhibitor of c-Met, SU11274 was first reported by Sattler, et al. (Pfizer; previously Sugen), in cells transformed by the oncogenic Tpr-Met as a model, as well as in SCLC (Sattler, et al., Cancer Res, 63, (17), 5462-9 (2003)). Recently, small molecular inhibitors of c-Met, such as APL-101 and Capmatilib, have shown promising efficacy in the clinic against lung cancers and brain tumors. However, clinical data indicates that many cancer patients are not responsive to c-Met inhibitors and the efficacy of c-Met inhibitors is limited. Therefore, there is an urgent need to develop new methods for treating cancer patients using c-Met inhibitors.

### SUMMARY

In one aspect, the present disclosure provides a method for predicting responsiveness of a subject having cancer to treatment with a c-Met inhibitor, said method comprising detecting a c-Met gene mutation, a c-Met gene fusion, a c-Met gene amplification, a c-Met expression level or a combination thereof in a cancer sample from a subject, and determining whether the cancer is likely to respond to treatment with the c-Met inhibitor. In one embodiment, the method comprises the steps of detecting an expression level of active c-Met in a cancer sample from a subject; detecting a c-Met gene mutation, a c-Met gene fusion or a c-Met gene amplification in the cancer sample; determining that the expression level of active c-Met is higher than a reference expression level of c-Met; and determining that the subject is likely to respond to treatment with the c-Met inhibitor.

In another aspect, the present disclosure provides a method for treating a subject having cancer, the method comprising: detecting a c-Met gene mutation, a c-Met gene fusion, a c-Met gene amplification, a c-Met expression level or a combination thereof in a cancer sample from a subject; determining whether the cancer is likely to respond to treatment with the c-Met inhibitor; and administering to the subject a c-Met inhibitor when the cancer is likely to respond to treatment with the c-Met inhibitor, and administering to the subject an anti-cancer agent other than a c-Met inhibitor when the cancer is not likely to respond to treatment with the c-Met inhibitor. In one embodiment, the method comprises the steps of detecting an expression level of active c-Met in a cancer sample from a subject; detecting a c-Met gene mutation, a c-Met gene fusion or a c-Met gene amplification in the cancer sample; determining that the expression level of active c-Met is higher than a reference expression level of c-Met; determining that the subject is likely to respond to treatment with a c-Met inhibitor; and administering to the subject the c-Met inhibitor.

In certain embodiment, the expression level of active c-Met is a mRNA level or a protein level. In certain embodiments, the active c-Met is a wild-type c-Met, a mutated c-Met, a c-Met fusion or a combination thereof.

In certain embodiments, the c-Met gene mutation results in a mutated c-Met protein with an amino acid change selected from the group consisting of K6N, V13L, G24E, E34A, E34K, A347T, M35V, A48G, H60Y, D94Y, G109R, S135N, D153A, H159R, E167K, E168D, E168K, T17I, P173A, R191W, S197F, T200A, A204PfsTer3, F206S, L211W, G212V, S213L, T222M, L238YfsTer25, S244Y, I259F, T273N, F281L, E293K, K305_R307del, A320V, S323G, G344R, M362T, N375K, N375S, V378I, H396Q, C397S, S406Ter, F430L, F445L, L455I, T457HfsTer21, P472S, E493K, Y501H, L515M, L530V, V546M, R547Q, S572N, R591W, K595T, R602K, L604I, L604V, T618M, T621I, M630T, M636V, I638L, G645R, T646A, T651S, G679V, R731Q, S752Y, F753C, P761S, V765D, K783E, F804C, R811H, E815D, T835PfsTer7, G843R, I852F, I852N, Y853H, D882N, D882Y, E891K, L905_H906delinsY, H906Y, V910F, Q931R, V937I, V941L, Q944Ter, L967F, R976T, L982_D1028del, R988C, Y989C, Y989Ter, A991P, T995N, V1007I, P1009S, T1010I, M10131, S1015Ter, D1028H, S1033L, R1040Q, Y1044C, Q1085K, G1120V, G1137A, L1158F, S1159L, R1166Q, R1166Ter, R1184Q, R1188Ter, D1198H, V1238I, A1239V, D1240N, Y1248H, A1299V, L1330YfsTer4, I316M, I333L, A1357V, V1368D, A1381T, L1386V and S1403Y and a combination thereof.

In certain embodiments, the c-Met gene fusion results in a gene fusion product selected from the group consisting of ACTG1/MET, ANXA2/MET, CAPZA2/MET, DNAL1/MET, FN1/MET, GTF2I/MET, KANK1/MET, MECP2/MET, MET/AGMO, MET/ANXA2, MET/CAPZA2, MET/CAV1, MET/IGF2, MET/INTU, MET/ITGA3, MET/NEDD4L, MET/PIEZO1, MET/PLEC, MET/POLR2A, MET/SLC16A3, MET/SMYD3, MET/ST7, MET/STEAP2-AS1, MET/TES, MET/TTC28-AS1, MGEA5/MET, PPM1G/MET, RPS27A/MET, ST7/MET, TES/MET, ZKSCAN1/MET and a combination thereof.

In certain embodiments, the cancer is selected from the groups consisting of lung cancer, melanoma, renal cancer, liver cancer, myeloma, prostate cancer, breast cancer, colorectal cancer, pancreatic cancer, thyroid cancer, hematological cancer, leukemia and non-Hodgkin's lymphoma.

In certain embodiments, the cancer is non-small cell lung cancer (NSCLC), renal cell carcinoma or hepatocellular carcinoma.

In certain embodiments, the cancer sample is tissue or blood.

In certain embodiments, the c-Met gene mutation, the c-Met gene fusion, or the c-Met gene amplification is detected using next generation sequencing.

In certain embodiments, the expression level of active c-Met is detected using an amplification assay, a hybridization assay, a sequencing assay, or an immunoassay.

In certain embodiments, the c-Met inhibitor is selected from the group consisting of Crizotinib, Cabozantinib, Tepotinib, AMG337 APL-101 (PLB1001, bozitinib), SU11274, PHA665752, K252a, PF-2341066, AM7, JNJ-38877605, PF-04217903, MK2461, GSK1363089 (XL880, foretinib), AMG458, Tivantinib (ARQ197), INCB28060 (INC280, capmatinib), E7050, BMS-777607, savolitinib (volitinib), HQP-8361, merestinib, ARGX-111, onartuzumab, rilotumumab, emibetuzumab, and XL184.

In certain embodiments, the c-Met inhibitor is an anti-c-Met antibody.

In certain embodiments, the c-Met inhibitor comprises a compound of the following formula

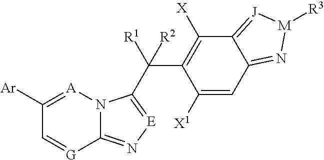

wherein:
R¹ and R² are independently hydrogen or halogen;
X and X¹ are independently hydrogen or halogen;
A and G are independently CH or N, or CH═G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, -CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH- C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl; and
R⁷ and R⁸ are independently hydrogen or C₁₋₆alkyl.

### DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the effect of APL-101 on LU0858 PDX model.

FIG. 2 shows the effect of APL-101 on LU1902 PDX model.

FIG. 3 shows the effect of APL-101 on LU2503 PDX model.

FIG. 4 shows the effect of APL-101 on MKN45 CDX model.

FIG. 5 shows the protein expression of c-Met and fusion derivative in different tumor cell lines as measured via Western blot. A549 was included as a negative control as the c-Met expression in this cell line is known to be very low.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the biological and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

As used herein, the term “administering” means providing a pharmaceutical agent or composition to a subject, and includes, but is not limited to, administering by a medical professional and self-administering.

As used herein, an “antibody” encompasses naturally occurring immunoglobulins as well as non-naturally occurring immunoglobulins, including, for example, single chain antibodies, chimeric antibodies (e.g., humanized murine antibodies), and heteroconjugate antibodies (e.g., bispecific antibodies). Fragments of antibodies include those that bind antigen, (e.g., Fab′, F(ab′)2, Fab, Fv, and rIgG). See also, e.g., Pierce Catalog and Handbook, 1994-1995 (Pierce Chemical Co., Rockford, Ill.); Kuby, J., Immunology, 3rd Ed., W.H. Freeman & Co., New York (1998). The term antibody also includes bivalent or bispecific molecules, diabodies, triabodies, and tetrabodies. The term “antibody” further includes both polyclonal and monoclonal antibodies.

As used herein, the term “cancer” refers to any diseases involving an abnormal cell growth and includes all stages and all forms of the disease that affects any tissue, organ or cell in the body. The term includes all known cancers and neoplastic conditions, whether characterized as malignant, benign, soft tissue, or solid, and cancers of all stages and grades including pre- and post-metastatic cancers. In general, cancers can be categorized according to the tissue or organ from which the cancer is located or originated and morphology of cancerous tissues and cells. As used herein, cancer types include, acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, breast cancer, Burkitt's lymphoma, cerebellar astrocytoma, cerebral astrocytoma/malignant glioma, cervical cancer, chronic lymphocytic leukemia, chronic myelogenous leukemia, colon cancer, emphysema, endometrial cancer, ependymoma, esophageal cancer, Ewing family of tumors, Ewing's sarcoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukaemia, liver cancer, lung cancer, medulloblastoma, melanoma, neuroblastoma, non-Hodgkin lymphoma, ovarian cancer, pancreatic cancer, pharyngeal cancer, prostate cancer, rectal cancer, renal cell carcinoma (kidney cancer), retinoblastoma, skin cancer, stomach cancer, supratentorial primitive neuroectodermal tumors, testicular cancer, throat cancer, thyroid cancer, vaginal cancer, visual pathway and hypothalamic glioma.

The term “cancer sample” includes a biological sample or a sample from a biological source that contains one or more cancer cells. Biological samples include samples from body fluids, e.g., blood, plasma, serum, or urine, or samples derived, e.g., by biopsy, from cells, tissues or organs, preferably tumor tissue suspected to include or essentially consist of cancer cells.

The term “c-Met” refers to a proto-oncogene that encodes a protein known as hepatocyte growth factor receptor (HGFR). c-Met protein is composed of the α chain and β chain generated by cleaving a precursor of c-Met (pro c-Met) and forms a dimer by a disulfide linkage. c-Met is a receptor penetrating a cell membrane and the entire α chain and a part of the β chain are present extracellularly (see, e.g., Mark, et al., The Journal of Biological Chemistry (1992) 267:26166-71; Ayumi I, Journal of Clinical and Experimental Medicine (2008) 224:51-55). See also GenBank Accession No: NP_000236.2 for human c-Met and its α chain and β chain. It has been shown that abnormal c-Met activation in cancer correlates with poor prognosis, where aberrantly active c-Met triggers tumor growth, formation of new blood vessels that supply the tumor with nutrients, and cancer spread or other organs.

The term “active c-Met” refers to a protein having the catalytic domain of c-Met or a nucleotide encoding the same. An active c-Met can be a wild type c-Met protein. In certain embodiments, an active c-Met can be a mutated c-Met protein but retains the catalytic activity as the wild type c-Met protein. In certain embodiments, an active c-Met can be a c-Met fusion protein, e.g., a c-Met or a fragment thereof fused to a second protein, which retain the catalytic domain as the wild type c-Met protein. In certain embodiment, an active c-Met protein may have increased catalytic activity compared to a wild type c-Met protein.

The term “c-Met alteration” or “c-Met gene alteration” as used herein refers an alteration of the nucleotide sequence of the c-Met gene in the genome of an organism or extrachromosomal DNA. A c-Met gene alteration includes substitution, deletion, and/or insertion of one or more nucleotides. For example, a c-Met gene alteration can be a c-Met gene mutation where one or more nucleotides are deleted from the c-Met gene, substituted for other nucleotides, or inserted into the c-Met gene. A c-Met gene alteration can also be a fusion where a fragment of the c-Met gene is fused to at least a fragment of another gene or another nucleotide sequence, or any combination of the above. A c-Met gene alteration also includes c-Met gene amplification where copy number of the c-Met gene increases.

A “c-Met inhibitor,” as used herein, refers an agent that can suppress the expression or activity of c-Met protein. Examples of c-Met inhibitor include, without limitation Crizotinib, Cabozantinib, Tepotinib, AMG337 APL-101 (PLB1001, bozitinib), SU11274, PHA665752, K252a, PF-2341066, AM7, JNJ-38877605, PF-04217903, MK2461, GSK1363089 (XL880, foretinib), AMG458, Tivantinib (ARQ197), INCB28060 (INC280, capmatinib), E7050, BMS-777607, savolitinib (volitinib), HQP-8361, merestinib, ARGX-111, onartuzumab, rilotumumab, emibetuzumab XL184 and compounds disclosed in US20150218171.

The term “complementarity” refers to the ability of a nucleic acid to form hydrogen bond(s) with another nucleic acid sequence by either traditional Watson-Crick or other non- traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60% >, 70% >, 80% >, 90%, and 100% complementary).

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps.

The terms “determining,” “assessing,” “measuring” and “detecting” can be used interchangeably and refer to both quantitative and semi-quantitative determinations. Where either a quantitative and semi-quantitative determination is intended, the phrase “determining a level” of a polynucleotide or polypeptide of interest or “detecting” a polynucleotide or polypeptide of interest can be used.

The term “hybridizing” refers to the binding, duplexing, or hybridizing of a nucleic acid molecule preferentially to a particular nucleotide sequence under stringent conditions. The term “stringent conditions” refers to hybridization and wash conditions under which a probe will hybridize preferentially to its target subsequence, and to a lesser extent to, or not at all to, other sequences in a mixed population (e.g., a cell lysate or DNA preparation from a tissue biopsy). A stringent condition in the context of nucleic acid hybridization are sequence dependent, and are different under different environmental parameters. An extensive guide to the hybridization of nucleic acids is found in, e.g., Tijssen Laboratory Techniques in Biochemistry and Molecular Biology-Hybridization with Nucleic Acid Probes part I, Ch. 2, “Overview of principles of hybridization and the strategy of nucleic acid probe assays,” (1993) Elsevier, N.Y. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook and Russell Molecular Cloning: A Laboratory Manual (3rd ed.) Vol. 1-3 (2001) Cold Spring Harbor Laboratory, Cold Spring Harbor Press, NY). An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4×SSC to 6×SSC at 40° C. for 15 minutes.

The term “gene product” or “gene expression product” refers to an RNA or protein encoded by the gene.

The term “c-Met expression level” and “expression level of c-Met” refer to the amount or quantity of c-Met expression present in a sample. Such amount or quantity may be expressed in the absolute terms, i.e., the total quantity of c-Met expression in the sample, or in the relative terms, i.e., the concentration or percentage of the c-Met in the sample. Level of c-Met expression can be measured at RNA level (for example as mRNA amount or quantity), or at protein level (for example as protein or protein complex amount or quantity). In certain embodiments, the c-Met expression level can be measured at a subset of c-Met protein level, for example, the level of phosphorylated c-Met protein.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

The term “responsive” or “responsiveness” as used in the context of a patient's response to a cancer therapy, are used interchangeably and refer to a beneficial patient response to a treatment as opposed to unfavorable responses, i.e. adverse events. In a patient, beneficial response can be expressed in terms of a number of clinical parameters, including loss of detectable tumor (complete response), decrease in tumor size and/or cancer cell number (partial response), tumor growth arrest (stable disease), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment, and therefore decreased responsiveness.

As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “sample” as used herein refers to a biological sample that is obtained from a subject and contains one or more c-MET gene alteration of interest. Examples of sample include, without limitation, bodily fluid, such as blood, plasma, serum, urine, vaginal fluid, uterine or vaginal flushing fluids, plural fluid, ascitic fluid, cerebrospinal fluid, saliva, sweat, tears, sputum, bronchioalveolar lavage fluid, etc., and tissues, such as biopsy tissue (e.g. biopsied bone tissue, bone marrow, breast tissue, gastrointestinal tract tissue, lung tissue, liver tissue, prostate tissue, brain tissue, nerve tissue, meningeal tissue, renal tissue, endometrial tissue, cervical dittuse, lymph node tissue, muscle tissue, or skin tissue), a paraffin embedded tissue. In certain embodiments, the sample can be a biological sample comprising cancer cells. In some embodiments, the sample is a fresh or archived sample obtained from a tumor, e.g., by a tumor biopsy or fine needle aspirate. The sample also can be any biological fluid containing cancer cells. The collection of a sample from a subject is performed in accordance with the standard protocol generally followed by hospital or clinics, such as during a biopsy.

The term “treatment,” “treat,” or “treating” refers to a method of reducing the effects of a cancer (e.g., breast cancer, lung cancer, ovarian cancer or the like) or symptom of cancer. Thus, in the disclosed method, treatment can refer to a 10%, 20%, 30%, 40%, 50%, 60%, 70%, 80%, 90%, or 100% reduction in the severity of a cancer or symptom of the cancer. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

### c-Met Gene Alterations

The methods and compositions described herein are based, in part, on the discovery of c-Met gene alterations whose presence in cancer samples is indicative of responsiveness of cancer patients to a c-Met inhibitor. In certain embodiments, the c-Met gene alterations include, without limitation, c-Met gene mutation, c-Met gene fusion and c-Met gene amplification.

The proto-oncogene c-MET encodes for the receptor tyrosine kinase (RTK) c-Met. Cells of epithelial-endothelial origin widely express c-MET, where it is essential for embryonic development and tissue repair. Hepatocyte growth factor (HGF) is the only known ligand for the c-Met receptor and is expressed mainly in cells of mesenchymal origin. Under normal conditions, c-Met dimerizes and autophosphorylates upon ligand binding, which in turn creates active docking sites for proteins that mediate downstream signaling leading to the activation of the mitogen-activated protein kinase (MAPK), phosphatidylinositol 3-kinase (PI3K)-AKT, v-src sarcoma viral oncogene homolog (SRC), signal transducer and activator of transcription (STAT) signaling pathways. Such activation evokes a variety of pleiotropic biological responses leading to increased cell growth, scattering and motility, invasion, protection from apoptosis, branching morphogenesis, and angiogenesis. However, under pathological conditions improper activation of c-Met may confer proliferative, survival and invasive/metastatic abilities of cancer cells.

Deregulation and the consequent aberrant signaling of c-Met may occur by different mechanisms including gene amplification and activating mutations. It has been reported that c-Met is overexpressed in a variety of carcinomas including lung, breast, ovary, kidney, colon, thyroid, live rand gastric carcinomas. Such overexpression could be the result of transcription activation, hypoxia-induced overexpression, or as a result of c-Met gene amplification. While gene amplification is a frequent genetic alteration of c-Met and has been reported as associated with a poor prognosis in NSCLC, colorectal and gastric cancer, oncogenic mutations on the c-Met gene are rarely found in patients with nonhereditary cancer. Potential oncogenic mutations involve mainly point mutations that generate an alternative splicing encoding a shorter protein that lacks exon 14, which encodes for juxtamembrane domain of c-Met; point mutations in the kinase domain that render the enzyme constitutively active; and Y1003 mutations that inactivate the Cb1 binding site leading to constitutive c-Met expression. In contrast, several other mutations (i.e., N375S, R988C and T1010I) have been reported as SNPs since they have been found to lack transforming abilities. In the present disclosure, the inventors have surprisingly found that some c-MET gene alterations are indicative of responsiveness when the cancer patients are treated with a c-Met inhibitor.

In certain embodiments, the c-Met gene alteration disclosed herein results in the skipping of exon 14 of the c-Met gene during transcription.

In certain embodiments, the c-Met gene alteration disclosed herein is a c-Met gene mutation which results in a mutated c-Met protein with an amino acid change shown in Table 1.

The inventor of the present disclosure also surprisingly found that some alterations of c-Met gene that results in a c-Met gene fusion are indicative of responsiveness of a cancer patient being treated with a c-Met inhibitor.

“Gene fusion” as used herein refers to a chimeric genomic DNA, a chimeric messenger RNA, a truncated protein or a chimeric protein resulting from the fusion of at least a portion of a first gene to at least a portion of a second gene. The gene fusion need not include entire genes or exons of genes.

In certain embodiments, the c-Met gene fusion results in a gene fusion product shown in Table 2.

The gene fusion product “ACTG1/MET” used herein means that the upstream gene ACTG1 is fused with the downstream gene MET. Other gene fusion product with the similar expression can be explained likewise.

“c-Met gene amplification” refers to copy number increase of g-Met gene in a cell. In certain embodiments, c-Met gene amplification results in overexpression of c-Met gene.

### Combinatory c-Met Biomarkers

The present disclosure in one aspect relates to the use of multiple c-Met related biomarkers in cancer treatment. In certain embodiments, the presence of multiple c-Met related biomarkers indicates an enhanced responsiveness of a subject having cancer to a c-Met inhibitor. In certain embodiments, the c-Met related biomarkers include c-Met gene mutation, c-Met gene fusion, c-Met gene amplification, and a c-Met expression level.

In certain embodiment, the presence of both increased expression of active c-Met and at least one c-Met gene alteration, such as a c-Met gene mutation, a c-Met gene fusion and a c-Met gene amplification, indicates an increased response to a c-Met inhibitor. In such case, the present disclosure provides a method for treating a subject having cancer comprising: detecting both an increased expression level of active c-Met and a c-Met gene alteration selected from a c-Met gene mutation, a c-Met gene fusion and a c-Met gene amplification in a cancer sample from a subject; and administering to the subject a c-Met inhibitor. In certain embodiment, a combination of increased expression level of active c-Met and a c-Met gene alteration in the cancer indicates that the cancer has deregulated c-Met activity as well as genomic instability. In certain embodiment, a combination of increased expression level of active c-Met and a c-Met gene alteration in the cancer indicates that deregulated c-Met activity is the driver of the cancer, which renders the cancer susceptible to c-Met inhibitor.

In certain embodiments, the presence of both a c-Met gene mutation and a c-Met gene amplification indicates an increased response to a c-Met inhibitor. In such case, the present disclosure provides a method for treating a subject having cancer comprising: detecting both a c-Met gene mutation and a c-Met gene amplification in a cancer sample from a subject; and administering to the subject a c-Met inhibitor. In certain embodiment, the c-Met gene mutation results in an exon 14 skipping.

In certain embodiments, the presence of both a c-Met gene mutation and an increased c-Met expression level indicates an increased response to a c-Met inhibitor. In such case, the present disclosure provides a method for treating a subject having cancer comprising: detecting both a c-Met gene mutation and an increased c-Met expression level in a cancer sample from a subject; and administering to the subject a c-Met inhibitor. In certain embodiment, the c-Met gene mutation results in an exon 14 skipping. In certain embodiments, the increased c-Met expression level results in an increased level of c-Met protein. In certain embodiments, the increased c-Met expression level is an increased phosphorylation of c-Met protein.

In certain embodiments, the presence of both a c-Met gene amplification and an increased c-Met expression level indicates an increased response to a c-Met inhibitor. In such case, the present disclosure provides a method for treating a subject having cancer comprising: detecting both a c-Met gene amplification and an increased c-Met expression level in a cancer sample from a subject; and administering to the subject a c-Met inhibitor. In certain embodiments, the increased c-Met expression level results in an increased level of c-Met protein. In certain embodiments, the increased c-Met expression level is an increased phosphorylation of c-Met protein.

In certain embodiments, the presence of at least two c-Met gene mutations indicates an increase response to a c-Met inhibitor. In such case, the present disclosure provides a method for treating a subject having cancer comprising: detecting at least two c-Met gene mutations described herein in a cancer sample from a subject; and administering to the subject a c-Met inhibitor. In certain embodiments, one of the at least two c-Met gene mutations results in an exon 14 skipping.

In certain embodiments, the presence of both a c-Met gene mutation and a c-Met gene fusion indicates an increased response to a c-Met inhibitor. In such case, the present disclosure provides a method for treating a subject having cancer comprising: detecting both a c-Met gene mutation and a c-Met gene fusion in a cancer sample from a subject; and administering to the subject a c-Met inhibitor. In certain embodiment, the c-Met gene mutation results in an exon 14 skipping.

In certain embodiments, the presence of both a c-Met gene fusion and a c-Met gene amplification indicates an increased response to a c-Met inhibitor. In such case, the present disclosure provides a method for treating a subject having cancer comprising: detecting both a c-Met gene fusion and a c-Met gene amplification in a cancer sample from a subject; and administering to the subject a c-Met inhibitor.

In certain embodiments, the presence of both a c-Met gene fusion and an increased c-Met expression level indicates an increased response to a c-Met inhibitor. In such case, the present disclosure provides a method for treating a subject having cancer comprising: detecting both a c-Met gene fusion and an increased c-Met expression level in a cancer sample from a subject; and administering to the subject a c-Met inhibitor. In certain embodiments, the increased c-Met expression level results in an increased level of c-Met protein. In certain embodiments, the increased c-Met expression level is an increased phosphorylation of c-Met protein.

In certain embodiments, the presence of multiple c-Met related biomarkers in a subject having cancer indicates that the subject has at least 50%, 55%, 60%, 65%, 70%, 75%, 80%, 85%, 90%, 95%, 96%, 97%, 98% or 99% of chance to respond to a treatment of c-Met inhibitor.

### Detection Reagents for c-Met Gene Alteration or c-Met Gene Expression

In one aspect, the present disclosure provides detection reagents for detecting the c-Met gene alteration or c-Met gene expression disclosed herein.

In certain embodiments, the detection reagents comprise primers or probes that can hybridize to the polynucleotide of the c-Met gene or c-Met mRNA.

The term “primer” as used herein refers to oligonucleotides that can specifically hybridize to a target polynucleotide sequence, due to the sequence complementarity of at least part of the primer within a sequence of the target polynucleotide sequence. A primer can have a length of at least 8 nucleotides, typically 8 to 70 nucleotides, usually of 18 to 26 nucleotides. For proper hybridization to the target sequence, a primer can have at least 75%, at least 80%, at least 85%, at least 90%, or at least 95% sequence complementarity to the hybridized portion of the target polynucleotide sequence. Oligonucleotides useful as primers may be chemically synthesized according to the solid phase phosphoramidite triester method first described by Beaucage and Caruthers, Tetrahedron Letts. (1981) 22: 1859-1862, using an automated synthesizer, as described in Needham-Van Devanter et al, Nucleic Acids Res. (1984) 12:6159-6168.

Primers are useful in nucleic acid amplification reactions in which the primer is extended to produce a new strand of the polynucleotide. Primers can be readily designed by a skilled artisan using common knowledge known in the art, such that they can specifically anneal to the nucleotide sequence of the target nucleotide sequence of the c-Met gene mutation or gene fusion provided herein. Usually, the 3′ nucleotide of the primer is designed to be complementary to the target sequence at the corresponding nucleotide position, to provide optimal primer extension by a polymerase.

The term “probe” as used herein refers to oligonucleotides or analogs thereof that can specifically hybridize to a target polynucleotide sequence, due to the sequence complementarity of at least part of the probe within a sequence of the target polynucleotide sequence. Exemplary probes can be, for example DNA probes, RNA probes, or protein nucleic acid (PNA) probes. A probe can have a length of at least 8 nucleotides, typically 8 to 70 nucleotides, usually of 18 to 26 nucleotides. For proper hybridization to the target sequence, a probe can have at least 75%, at least 80%, at least 85%, at least 90%, or at least 95% sequence complementarity to hybridized portion of the target polynucleotide sequence. Probes and also be chemically synthesized according to the solid phase phosphoramidite triester method as described above. Methods for preparation of DNA and RNA probes, and the conditions for hybridization thereof to target nucleotide sequences, are described in Molecular Cloning: A Laboratory Manual, J. Sambrook et al., eds., 2nd edition. Cold Spring Harbor Laboratory Press, 1989, Chapters 10 and 11.

In certain embodiments, the primers and the probes provided herein are detectably labeled. Examples of the detectable label suitable for labeling primers and probes include, for example, chromophores, radioisotopes, fluorophores, chemiluminescent moieties, particles (visible or fluorescent), nucleic acids, ligand, or catalysts such as enzymes.

In certain embodiments, the detection reagents comprise an antibody that specifically binds to the c-Met protein.

The term “antibody” as used herein refers to an immunoglobulin or an antigen-binding fragment thereof, which can specifically bind to a target protein antigen. Antibodies can be identified and prepared by selection of antibodies from libraries of recombinant antibodies in phage or similar vectors, as well as preparation of polyclonal and monoclonal antibodies by immunizing animals such as rabbits or mice (see, e.g., Huse et al., Science (1989) 246:1275-1281; Ward et al, Nature (1989) 341 :544-546).

It can be understood that in certain embodiments, the antibodies are modified or labeled to be properly used in various detection assays. In certain embodiments, the antibody is detectably labeled.

### Sample Preparation

Any biological sample suitable for conducting the methods provided herein can be obtained from the subject. In certain embodiments, the sample can be further processed by a desirable method for performing the detection of the c-Met gene alteration.

In certain embodiments, the method further comprises isolating or extracting cancer cell (such as circulating tumor cell) from the biological fluid sample (such as peripheral blood sample) or the tissue sample obtained from the subject. The cancer cells can be separated by immunomagnetic separation technology such as that available from Immunicon (Huntingdon Valley, Pa.).

In certain embodiments, a tissue sample can be processed to perform in situ hybridization. For example, the tissue sample can be paraffin-embedded before fixing on a glass microscope slide, and then deparaffinized with a solvent, typically xylene.

In certain embodiments, the method further comprises isolating the nucleic acid, e.g. DNA or RNA from the sample. Various methods of extraction are suitable for isolating the DNA or RNA from cells or tissues, such as phenol and chloroform extraction, and various other methods as described in, for example, Ausubel et al., Current Protocols of Molecular Biology (1997) John Wiley & Sons, and Sambrook and Russell, Molecular Cloning: A Laboratory Manual 3^{rd} ed. (2001).

Commercially available kits can also be used to isolate DNA and/or RNA, including for example, the NucliSens extraction kit (Biomerieux, Marcy l'Etoile, France), QIAamp™ mini blood kit, Agencourt Genfind™, Rneasy® mini columns (Qiagen), PureLink® RNA mini kit (Thermo Fisher Scientific), and Eppendorf Phase Lock Gels™. A skilled person can readily extract or isolate RNA or DNA following the manufacturer's protocol.

### Methods of Detecting c-Met Gene Alteration or c-Met Expression Level

The methods of the present disclosure include detecting the c-Met gene alteration or c-Met expression level described herein in a sample obtained from a subject having cancer or suspected of having cancer. The c-Met gene alteration, such as c-Met gene mutation, c-Met gene fusion or c-Met gene amplification can be detected in the level of DNA (e.g. genomic DNA) or RNA (e.g. mRNA) using proper methods known in the art including, without limitation, amplification assay, hybridization assay, and sequencing assay. The c-Met expression level can be detected in the RNA (e.g. mRNA) level or protein level using proper methods known in the art including, without limitation, amplification assay, hybridization assay, sequencing assay, and immunoassay.

Amplification Assay

A nucleic acid amplification assay involves copying a target nucleic acid (e.g. DNA or RNA), thereby increasing the number of copies of the amplified nucleic acid sequence. Amplification may be exponential or linear. Exemplary nucleic acid amplification methods include, but are not limited to, amplification using the polymerase chain reaction (“PCR”, see U.S. Pat. Nos. 4,683,195 and 4,683,202; PCR Protocols: A Guide To Methods And Applications (Innis et al., eds, 1990)), reverse transcriptase polymerase chain reaction (RT-PCR), quantitative real-time PCR (qRT-PCR); quantitative PCR, such as TaqMan®, nested PCR, ligase chain reaction (See Abravaya, K., et al., Nucleic Acids Research, 23:675-682, (1995), branched DNA signal amplification (see, Urdea, M. S., et al., AIDS, 7 (suppl 2):S11-S14, (1993), amplifiable RNA reporters, Q-beta replication (see Lizardi et al., Biotechnology (1988) 6: 1197), transcription-based amplification (see, Kwoh et al., Proc. Natl. Acad. Sci. USA (1989) 86: 1173-1177), boomerang DNA amplification, strand displacement activation, cycling probe technology, self-sustained sequence replication (Guatelli et al., Proc. Natl. Acad. Sci. USA (1990) 87:1874-1878), rolling circle replication (U.S. Pat. No. 5,854,033), isothermal nucleic acid sequence based amplification (NASBA), and serial analysis of gene expression (SAGE).

In certain embodiments, the nucleic acid amplification assay is a PCR-based method. PCR is initiated with a pair of primers that hybridize to the target nucleic acid sequence to be amplified, followed by elongation of the primer by polymerase which synthesizes the new strand using the target nucleic acid sequence as a template and dNTPs as building blocks. Then the new strand and the target strand are denatured to allow primers to bind for the next cycle of extension and synthesis. After multiple amplification cycles, the total number of copies of the target nucleic acid sequence can increase exponentially.

In certain embodiments, intercalating agents that produce a signal when intercalated in double stranded DNA may be used. Exemplary agents include SYBR GREEN™ and SYBR GOLD™. Since these agents are not template-specific, it is assumed that the signal is generated based on template-specific amplification. This can be confirmed by monitoring signal as a function of temperature because melting point of template sequences will generally be much higher than, for example, primer-dimers, etc.

In certain embodiments, a detectably labeled primer or a detectably labeled probe can be used, to allow detection of the c-Met gene alteration corresponding to that primer or probe. In certain embodiments, multiple labeled primers or labeled probes with different detectable labels can be used to allow simultaneous detection of multiple c-Met gene alteration.

Hybridization Assay

Nucleic acid hybridization assays use probes to hybridize to the target nucleic acid, thereby allowing detection of the target nucleic acid. Non-limiting examples of hybridization assay include Northern blotting, Southern blotting, in situ hybridization, microarray analysis, and multiplexed hybridization-based assays.

In certain embodiments, the probes for hybridization assay are detectably labeled. In certain embodiments, the nucleic acid-based probes for hybridization assay are unlabeled. Such unlabeled probes can be immobilized on a solid support such as a microarray, and can hybridize to the target nucleic acid molecules which are detectably labeled.

In certain embodiments, hybridization assays can be performed by isolating the nucleic acids (e.g. RNA or DNA), separating the nucleic acids (e.g. by gel electrophoresis) followed by transfer of the separated nucleic acid on suitable membrane filters (e.g. nitrocellulose filters), where the probes hybridize to the target nucleic acids and allows detection. See, for example, Molecular Cloning: A Laboratory Manual, J. Sambrook et al., eds., 2nd edition, Cold Spring Harbor Laboratory Press, 1989, Chapter 7. The hybridization of the probe and the target nucleic acid can be detected or measured by methods known in the art. For example, autoradiographic detection of hybridization can be performed by exposing hybridized filters to photographic film.

In some embodiments, hybridization assays can be performed on microarrays. Microarrays provide a method for the simultaneous measurement of the levels of large numbers of target nucleic acid molecules. The target nucleic acids can be RNA, DNA, cDNA reverse transcribed from mRNA, or chromosomal DNA. The target nucleic acids can be allowed to hybridize to a microarray comprising a substrate having multiple immobilized nucleic acid probes arrayed at a density of up to several million probes per square centimeter of the substrate surface. The RNA or DNA in the sample is hybridized to complementary probes on the array and then detected by laser scanning. Hybridization intensities for each probe on the array are determined and converted to a quantitative value representing relative levels of the RNA or DNA. See, U.S. Pat. Nos. 6,040,138, 5,800,992 and 6,020,135, 6,033,860, and 6,344,316.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device. Useful microarrays are also commercially available, for example, microarrays from Affymetrix, from Nano String Technologies, QuantiGene 2.0 Multiplex Assay from Panomics.

In certain embodiments, hybridization assays can be in situ hybridization assay. In situ hybridization assay is useful to detect the presence of c-Met gene amplification. Probes useful for in situ hybridization assay can be mutation or gene fusion specific probes, which hybridize to a specific c-Met gene mutation or gene fusion to detect the presence or absence of the specific mutation or gene fusion of interest. Methods for use of unique sequence probes for in situ hybridization are described in U.S. Pat. No. 5,447,841, incorporated herein by reference. Probes can be viewed with a fluorescence microscope and an appropriate filter for each fluorophore, or by using dual or triple band-pass filter sets to observe multiple fluorophores. See, e.g., U.S. Pat. No. 5,776,688 to Bittner, et al., which is incorporated herein by reference. Any suitable microscopic imaging method can be used to visualize the hybridized probes, including automated digital imaging systems. Alternatively, techniques such as flow cytometry can be used to examine the hybridization pattern of the probes.

Sequencing Methods

Sequencing methods useful in the measurement of the c-Met gene alteration involves sequencing of the target nucleic acid. Any sequencing known in the art can be used to detect the c-Met gene alteration of interest. In general, sequencing methods can be categorized to traditional or classical methods and high throughput sequencing (next generation sequencing). Traditional sequencing methods include Maxam-Gilbert sequencing (also known as chemical sequencing) and Sanger sequencing (also known as chain-termination methods).

High throughput sequencing, or next generation sequencing, by using methods distinguished from traditional methods, such as Sanger sequencing, is highly scalable and able to sequence the entire genome or transcriptome at once. High throughput sequencing involves sequencing-by-synthesis, sequencing-by-ligation, and ultra-deep sequencing (such as described in Marguiles et al., Nature 437 (7057): 376-80 (2005)). Sequence-by-synthesis involves synthesizing a complementary strand of the target nucleic acid by incorporating labeled nucleotide or nucleotide analog in a polymerase amplification. Immediately after or upon successful incorporation of a label nucleotide, a signal of the label is measured and the identity of the nucleotide is recorded. The detectable label on the incorporated nucleotide is removed before the incorporation, detection and identification steps are repeated. Examples of sequence-by-synthesis methods are known in the art, and are described for example in U.S. Pat. Nos. 7,056,676, 8,802,368 and 7,169,560, the contents of which are incorporated herein by reference. Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the Illumina® sequencing platform.

Pyrosequencing involves hybridizing the target nucleic acid regions to a primer and extending the new strand by sequentially incorporating deoxynucleotide triphosphates corresponding to the bases A, C, G, and T (U) in the presence of a polymerase. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

In certain embodiments, the c-Met gene mutation, gene fusion or gene amplification described herein is detected by whole transcriptome shotgun sequencing (RNA sequencing). The method of RNA sequencing has been described (see Wang Z, Gerstein M and Snyder M, Nature Review Genetics (2009) 10:57-63; Maher C A et al., Nature (2009) 458:97-101; Kukurba K & Montgomery SB, Cold Spring Harbor Protocols (2015) 2015(11):951-969).

Immunoassay

Immunoassays used herein typically involves using antibodies that specifically bind to c-Met protein. Such antibodies can be obtained using methods known in the art (see, e.g., Huse et al., Science (1989) 246:1275-1281; Ward et al, Nature (1989) 341:544-546), or can be obtained from commercial sources. Examples of immunoassays include, without limitation, Western blotting, enzyme-linked immunosorbent assay (ELISA), enzyme immunoassay (EIA), radioimmunoassay (RIA), immunoprecipitations, sandwich assays, competitive assays, immunofluorescent staining and imaging, immunohistochemistry (IHC), and fluorescent activating cell sorting (FACS). For a review of immunological and immunoassay procedures, see Basic and Clinical Immunology (Stites & Terr eds., 7^{th} ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Ten, eds., 7^{th} ed. 1991).

In certain embodiments, the c-Met expression level is measured as the level of a subset of c-Met protein, such as the level of modified c-Met protein, e.g. phosphorylated c-Met protein. In such cases, the c-Met expression level can be detected using antibodies that specifically bind to the modified c-Met protein.

Any of the assays and methods provided herein for the measurement of the c-Met expression level can be adapted or optimized for use in automated and semi-automated systems, or point of care assay systems.

The c-Met expression level described herein can be normalized using a proper method known in the art. For example, the c-Met expression level can be normalized to a standard level of a standard marker, which can be predetermined, determined concurrently, or determined after a sample is obtained from the subject. The standard marker can be run in the same assay or can be a known standard marker from a previous assay. For another example, the c-Met expression level can be normalized to an internal control which can be an internal marker, or an average level or a total level of a plurality of internal markers.

### Comparing with a Reference Level

In certain embodiments, the methods disclosed herein include a step of comparing the detected c-Met expression level to a reference c-Met level.

The term “reference c-Met level” refers to a level of c-Met expression that is representative of a reference sample. In certain embodiments, the reference sample is obtained from a healthy subject or tissue. In certain embodiments, the reference sample is a cancer or tumor tissue. In certain embodiments, the reference c-Met level is obtained using the same or comparable measurement method or assay as used in the detection of the c-Met expression level in the test sample.

In certain embodiments, the reference c-Met level can be predetermined. For example, the reference c-Met level can be calculated or generalized based on measurements of the c-Met level in a collection of general cancer or tumor samples or tissues from a tumor of the same type, or from blood cancer. For another example, the reference c-Met level can be based on statistics of the level of the c-Met generally observed in an average cancer or tumor samples from a general cancer or tumor population.

In certain embodiments, the comparing step in the method provided herein involves determining the difference between the detected c-Met expression level and the reference c-Met level. The difference from the reference c-Met level can be elevation or reduction.

In certain embodiments, the difference from the reference c-Met level is further compared with a threshold. In certain embodiments, a threshold can be set by statistical methods, such that if the difference from the reference c-Met level reaches the threshold, such difference can be considered statistically significant. Useful statistical analysis methods are described in L. D. Fisher & G. vanBelle, Biostatistics: A Methodology for the Health Sciences (Wiley-Interscience, NY, 1993). Statistically significance can be determined based on confidence (“p”) values, which can be calculated using an unpaired 2-tailed t test. A p value less than or equal to, for example, 0.1, 0.05, 0.025, or 0.01 usually can be used to indicated statistical significance. Confidence intervals and p-values can be determined by methods well-known in the art. See, e.g., Dowdy and Wearden, Statistics for Research, John Wiley & Sons, New York, 1983.

### Treatment with c-Met Inhibitors

In another aspect, the present disclosure provides a method for treating a subject having cancer. In certain embodiments, the method comprises: detecting a c-Met gene mutation, a c-Met gene fusion, a c-Met gene amplification or a combination thereof in a cancer sample from a subject, and administering to the subject a c-Met inhibitor. In certain embodiments, the method comprises: detecting an expression level of active c-Met in a cancer sample from a subject; detecting a c-Met gene mutation, a c-Met gene fusion or a c-Met gene amplification in the cancer sample; determining that the expression level of active c-Met is higher than a reference expression level of c-Met; determining that the subject is likely to respond to treatment with a c-Met inhibitor; and administering to the subject the c-Met inhibitor.

In certain embodiments, c-Met inhibitor is selected from the group consisting of Crizotinib, Cabozantinib, Tepotinib, AMG337 APL-101 (PLB1001, bozitinib), SU11274, PHA665752, K252a, PF-2341066, AM7, JNJ-38877605, PF-04217903, MK2461, GSK1363089 (XL880, foretinib), AMG458, Tivantinib (ARQ197), INCB28060 (INC280, capmatinib), E7050, BMS-777607, savolitinib (volitinib), HQP-8361, merestinib, ARGX-111, onartuzumab, rilotumumab, emibetuzumab, and XL184.

In some embodiments, the c-Met inhibitor comprises a compound of the following formula

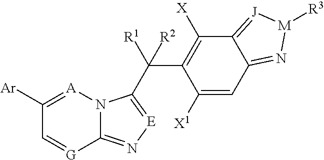

wherein:
R¹ and R² are independently hydrogen or halogen;
X and X¹ are independently hydrogen or halogen;
A and G are independently CH or N, or CH═G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl, halo C₁₋₆alkyl, halo C₁₋₆alkoxy, C₃₋₇cycloalkyl, halogen, cyano, amino, —CONR⁴R⁵, —NHCOR⁶, —SO₂NR⁷R⁸, C₁₋₆alkoxyl-, C₁₋₆alkyl-, amino-C₁₋₆alkyl-, heterocyclyl and heterocyclyl-C₁₋₆alkyl-, or two connected substituents together with the atoms to which they are attached form a 4-6 membered lactam fused with the aryl or heteroaryl;
R³ is hydrogen, C₁₋₆alkyl, C₁₋₆alkoxy, haloC₁₋₆alkyl, halogen, amino, or —CONH- C₁₋₆alkyl-heterocyclyl;
R⁴ and R⁵ are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl; and
R⁷ and R⁸ are independently hydrogen or C₁₋₆alkyl.

In some embodiments, the c-Met inhibitor is selected from the group consisting of:

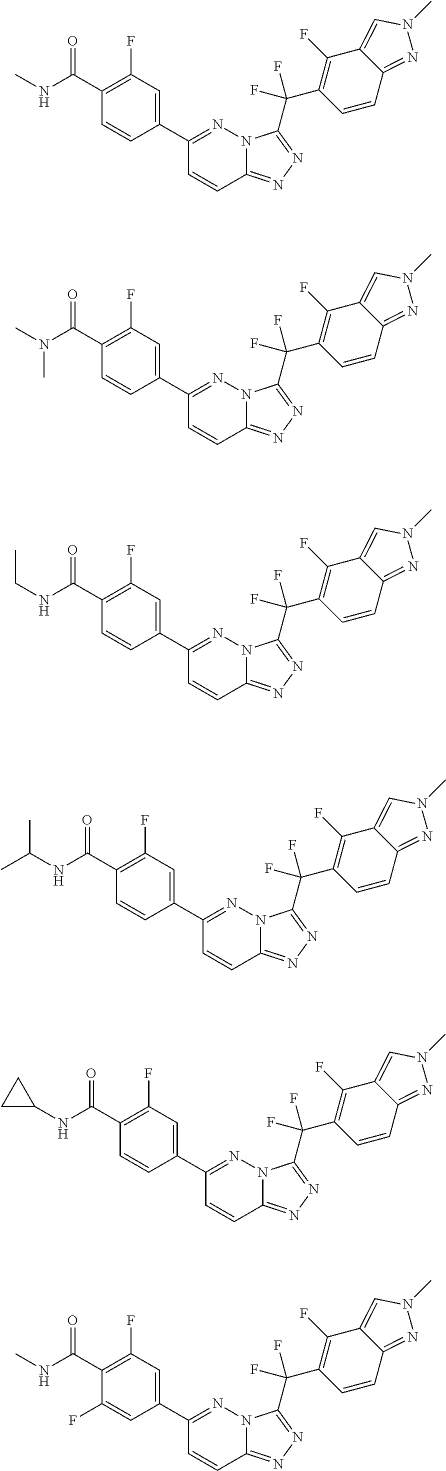

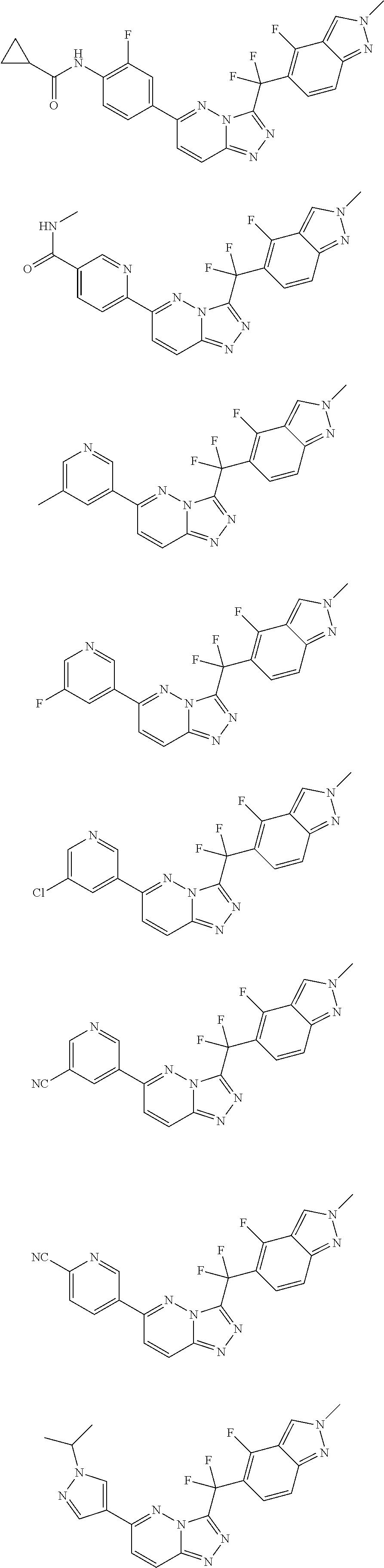

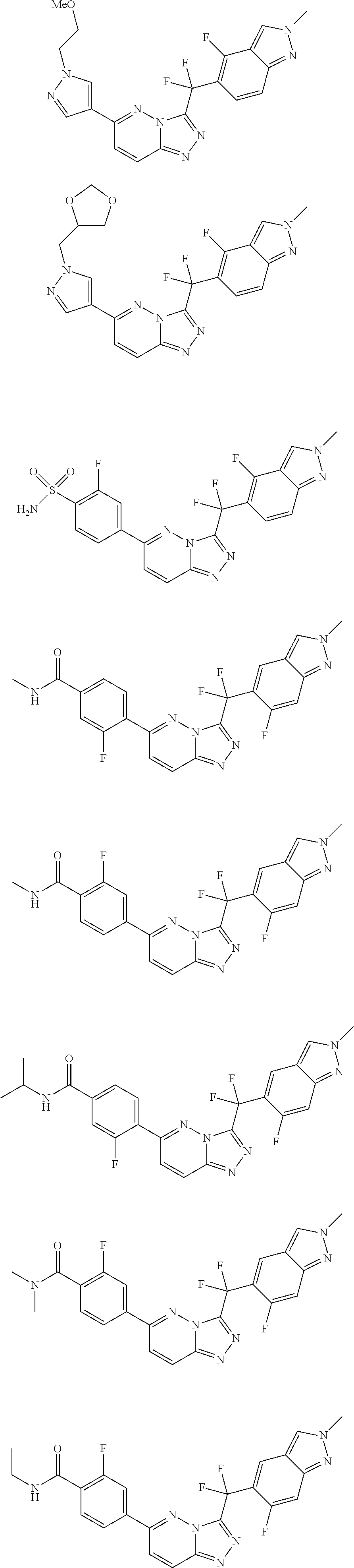

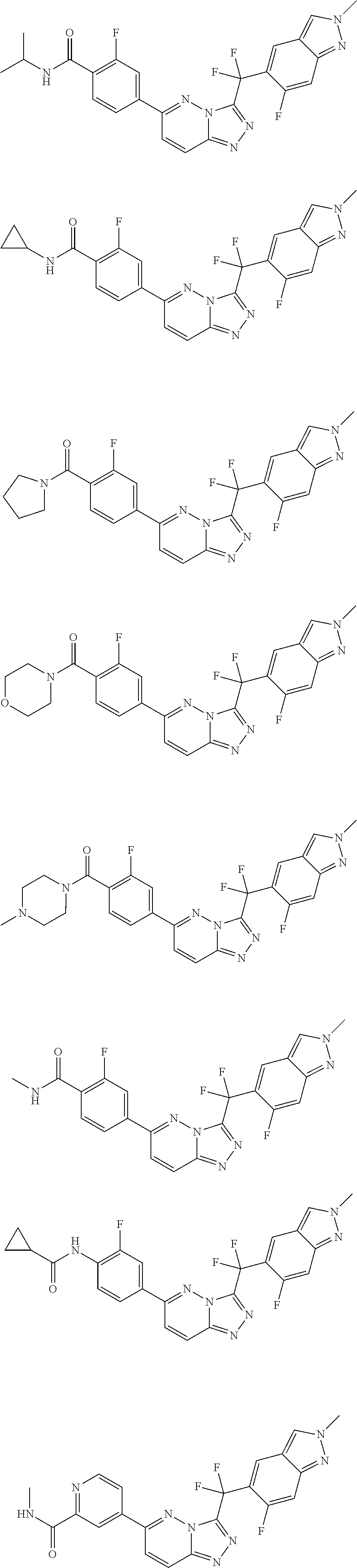

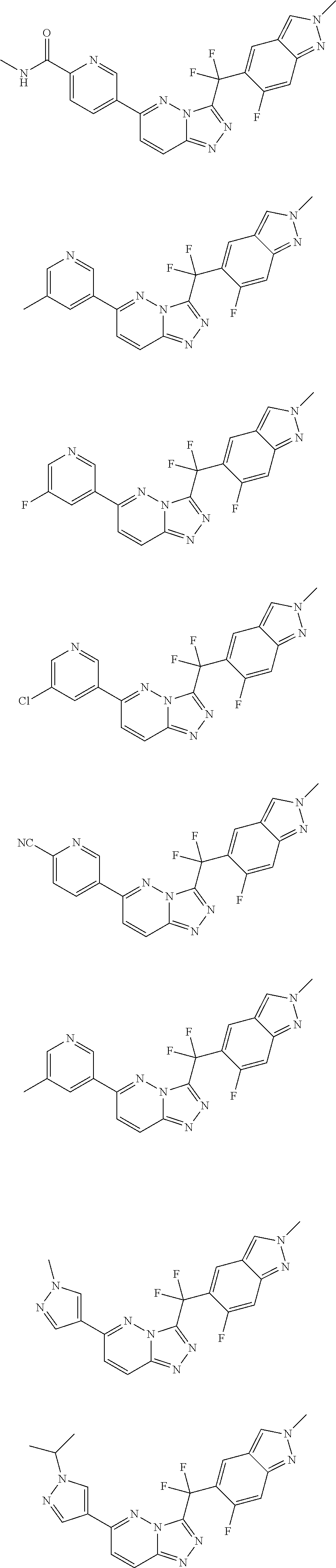

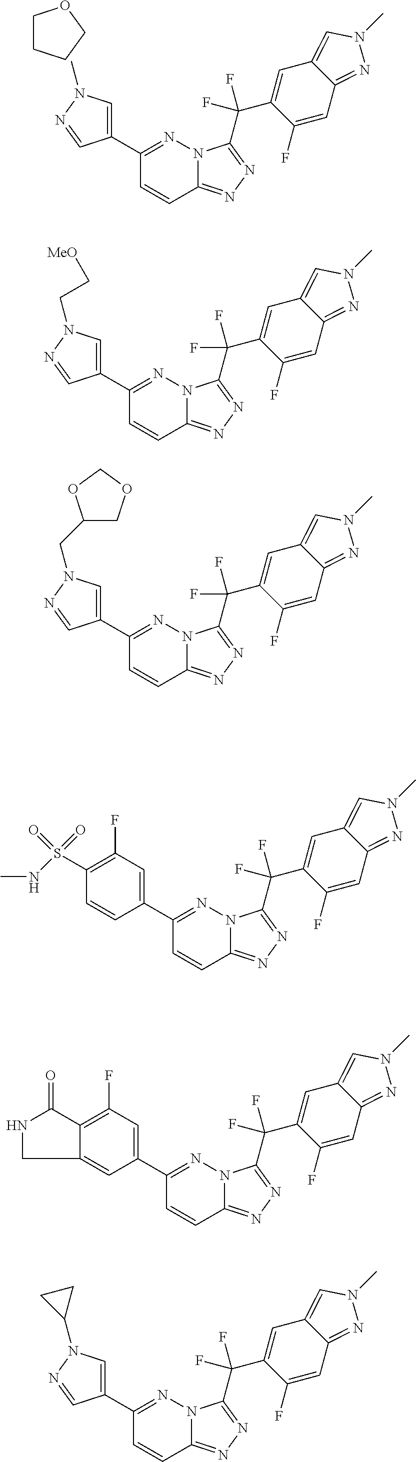

In certain embodiments, c-Met inhibitor is APL-101 (previously named CBT-101, see US20150218171, which is incorporated in its entirety by reference), which has the following formula:

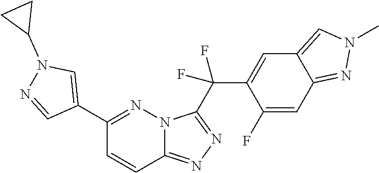 [SMILES: Cn1cc2cc(C(F)(F)c3nnc4ccc(-c5cnn(C6CC6)c5)nn34)c(F)cc2n1]

In certain embodiments, c-Met inhibitor can be formulated with a pharmaceutically acceptable carrier. The carrier, when present, can be blended with c-Met inhibitor in any suitable amounts, such as an amount of from 5% to 95% by weight of carrier, based on the total volume or weight of c-Met inhibitor and the carrier. In some embodiments, the amount of carrier can be in a range having a lower limit of any of 5%, 10%, 12%, 15%, 20%, 25%, 28%, 30%, 40%, 50%, 60%, 70% or 75%, and an upper limit, higher than the lower limit, of any of 20%, 22%, 25%, 28%, 30%, 40%, 50%, 60%, 70%, 75%, 80%, 85%, 90%, and 95%. The amount of carrier in a specific embodiment may be determined based on considerations of the specific dose form, relative amounts of c-Met inhibitor, the total weight of the composition including the carrier, the physical and chemical properties of the carrier, and other factors, as known to those of ordinary skill in the formulation art.

The c-Met inhibitor may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, the c-Met inhibitor may be administered in conjunction with other treatments. The c-Met inhibitor may be encapsulated or otherwise protected against gastric or other secretions, if desired.

A suitable, non-limiting example of a dosage of the c-Met inhibitor disclosed herein is from about 1 mg/kg to about 2400 mg/kg per day, such as from about 1 mg/kg to about 1200 mg/kg per day, 75 mg/kg per day to about 300 mg/kg per day, including from about 1 mg/kg to about 100 mg/kg per day. Other representative dosages of such agents include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. In some embodiments, the dosage of the c-Met inhibitor in human is about 400 mg/day given every 12 hours. In some embodiments, the dosage of the c-Met inhibitor in human ranges 300-500 mg/day, 100-600 mg/day or 25-1000 mg/day. The effective dose of c-Met inhibitor disclosed herein may be administered as two, three, four, five, six or more sub-doses, administered separately at appropriate intervals throughout the day.

### Anti-cancer Agents Other Than c-Met Inhibitor

The method of present disclosure also involves, after determining that a subject is not likely to respond to a c-Met inhibitor, administering to the subject an anti-cancer agent other than a c-Met inhibitor. These anti-cancer agents include, without limitation: alkylating agents or agents with an alkylating action, such as cyclophosphamide (CTX; e.g. cytoxan®), chlorambucil (CHL; e.g. leukeran®), cisplatin (CisP; e.g. platinol®) busulfan (e.g. myleran®), melphalan, carmustine (BCNU), streptozotocin, triethylenemelamine (TEM), mitomycin C, and the like; anti-metabolites, such as methotrexate (MTX), etoposide (VP16; e.g. vepesid®), 6-mercaptopurine (6MP), 6-thiocguanine (6TG), cytarabine (Ara-C), 5-fluorouracil (5-FU), capecitabine (e.g.Xeloda®), dacarbazine (DTIC), and the like; antibiotics, such as actinomycin D, doxorubicin (DXR; e.g. adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. taxol®) and pactitaxel derivatives, the cytostatic agents, glucocorticoids such as dexamethasone (DEX; e.g. decadron®) and corticosteroids such as prednisone, nucleoside enzyme inhibitors such as hydroxyurea, amino acid depleting enzymes such as asparaginase, leucovorin, folinic acid, raltitrexed, and other folic acid derivatives, and similar, diverse antitumor agents. The following agents may also be used as additional agents: amifostine (e.g. ethyol®), dactinomycin, mechlorethamine (nitrogen mustard), streptozocin, cyclophosphamide, lornustine (CCNU), doxorubicin lipo (e.g. doxil®), gemcitabine (e.g. gemzar®), daunorubicin lipo (e.g. daunoxome®), procarbazine, mitomycin, docetaxel (e.g. taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon alpha, interferon beta, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, and chlorambucil.

In certain embodiments, an anti-cancer agent other than a c-Met inhibitor is an anti-hormonal agent. As used herein, the term “anti-hormonal agent” includes natural or synthetic organic or peptide compounds that act to regulate or inhibit hormone action on tumors.

Anti-hormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. Fareston®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as Zoladex® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N6-(3-pyridinylcarbonyl)-L-lysyl-N6-(3-pyridinylcarbonyl)-D-lysyl-L-leucyl-N6-(1-methylethyl)-L-lysyl-L-proline (e.g Antide®, Ares-Serono); the LHRH antagonist ganirelix acetate; the steroidal anti-androgens cyproterone acetate (CPA) and megestrol acetate, commercially available as Megace® (Bristol-Myers Oncology); the nonsteroidal anti-androgen flutamide (2-methyl-N-[4, 20-nitro-3-(trifluoromethyl)phenylpropanamide), commercially available as Eulexin® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

In certain embodiments, an anti-cancer agent other than a c-Met inhibitor is an angiogenesis inhibitor. Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. Avastin™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. Vitaxin®); factors such as IFN-alpha (U.S. Pat. Nos. 41530,901, 4,503,035, and 5,231,176); angiostatin and plasminogen fragments (e.g. kringle 14, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloprotienase 2) inhibitors and MMP-9 (matrix-metalloprotienase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

In certain embodiments, an anti-cancer agent other than a c-Met inhibitor is a tumor cell pro-apoptotic or apoptosis-stimulating agent.

In certain embodiments, an anti-cancer agent other than a c-Met inhibitor is a signal transduction inhibitor. Signal transduction inhibitors include, for example: erbB2 receptor inhibitors, such as organic molecules, or antibodies that bind to the erbB2 receptor, for example, trastuzumab (e.g. Herceptin®); inhibitors of other protein tyrosine-kinases, e.g. imitinib (e.g. Gleevec®); ras inhibitors; raf inhibitors; MEK inhibitors; mTOR inhibitors; cyclin dependent kinase inhibitors; protein kinase C inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer); GW-282974 (Glaxo Wellcome plc); monoclonal antibodies such as AR-209 (Aronex Pharmaceuticals Inc. of The Woodlands, Tex., USA) and 2B-1 (Chiron); and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

In certain embodiments, an anti-cancer agent other than a c-Met inhibitor is a cancer immunotherapy agent, such as an antibody specifically binding to an immune checkpoint. Immune checkpoints include, for example: A2AR, B7.1, B7.2, B7-H2, B7-H3, B7-H4, B7-H6, BTLA, CD48, CD160, CD244, CTLA-4, ICOS, LAG-3, LILRB1, LILRB2, LILRB4, OX40, PD-1, PD-L1, PD-L2, SIRPalpha (CD47), TIGIT, TIM-3, TIM-1, TIM-4, and VISTA.

In certain embodiments, an anti-cancer agent other than a c-Met inhibitor is an anti-proliferative agent. Anti-proliferative agents include, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

This example illustrates that certain c-Met gene alterations can be used as biomarkers to determine a cancer is sensitive towards c-Met inhibitors.

### Methods

Cell lines and PDX models harboring c-Met point mutations and fusions were identified using the data in the public domain. To validate the c-Met fusion genes in the tumor cell lines, the fusion gene product (mRNA) was amplified using RT-PCR and cloned for Sanger sequencing. The expression of c-Met protein and c-Met fusion protein in tumor cell lines was validated using western blot. The levels of transcripts encoding c-Met protein and c-Met fusion proteins in tumor cell lines were measured using qRT-PCRPCT. A panel of identified cell lines with c-Met point mutations and fusions were then tested in vitro for their sensitivity towards APL101. PDX models with c-Met fusions and amplifications were treated with APL-101 to investigate the tumor's sensitivity towards the c-Met inhibitor in vivo.

### Results

A total of 976 cell lines and 1611 PDXs were screened for c-Met point mutations and fusions. For point mutations, recurrent mutations were selected and tested for IC50. As shown in Table 3, none of the 18 cell lines that harbor the point mutations but do not have c-Met amplification was sensitive towards APL-101. In contrast, the cell line HS 746.T, which harbors point mutation that causes exon 14 skipping and has c-Met gene amplification, was sensitive to APL-101. The expression of c-Met protein in HS746.T has been reported by Y. Asaoka et al. (Biochemical and Biophysical Research Communications (2010) 394:1042-1046).

For fusions, our analysis indicates an average of 1.16% of all analyzed tumor cell lines and models harboring a c-Met fusion mutation, 70% of which harboring a kinase-live fusion mutation (0.81% of all tumors analyzed) (see Table 6). The inventors identified a total of 26 c-Met fusion partners (see Table 8), and 37 different fusions events due to multiple fusion events involving a few recurrent partners. The fusions have been found in cancer types including cholangiocarcinoma, colorectal cancer, liver cancer, gastric cancer, lung cancer, etc., with lung cancer having the most events (see Table 9).

In order to illustrate the correlation between the efficacy of APL-101-treatment and the genotype as well as phenotype of c-MET alleles, the inventors identified the transcript sequences associated with the known fusion genes with c-MET as a partner and demonstrated the junction points in seven tumor cell lines. The inventors further measured the expression levels in transcripts and protein of c-Met and derivatives in selected cell lines using quantitative RT-PCR (qRT-PCR) and Western blot, respectively.

The inventors deployed 6 cell lines harboring recurrent fusions for in vitro sensitivity testing. Three of the cell lines, MKN45, MHCC97H, HCCLM3, all have fusions as well as c-Met amplification/overexpression, showed high sensitivity towards APL-101, with IC50s of 0.18, 0.24, and 0.61uM, respectively (see Table 11). The other three cell lines, which do not have high amplification of c-Met, were all unresponsive to APL-101, with IC50s higher than 10uM. The results demonstrated the correlation between the APL-101-sensitivity and the high expression in the transcription and protein levels of wild type c-MET alone or wild type c-MET together with one or more fusion genes each encoding an intact MET-derived protein kinase domain.

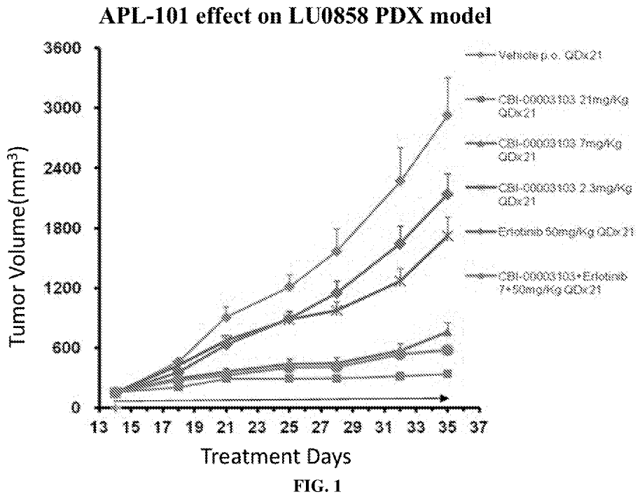

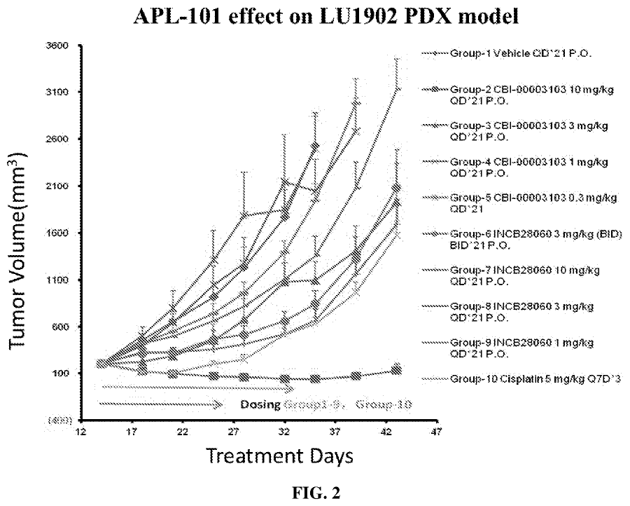

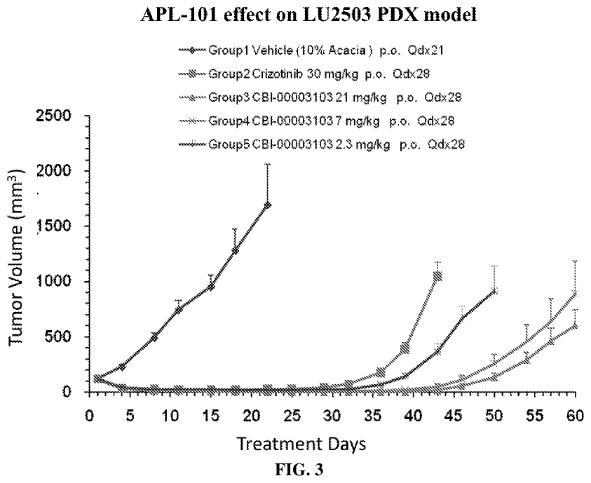

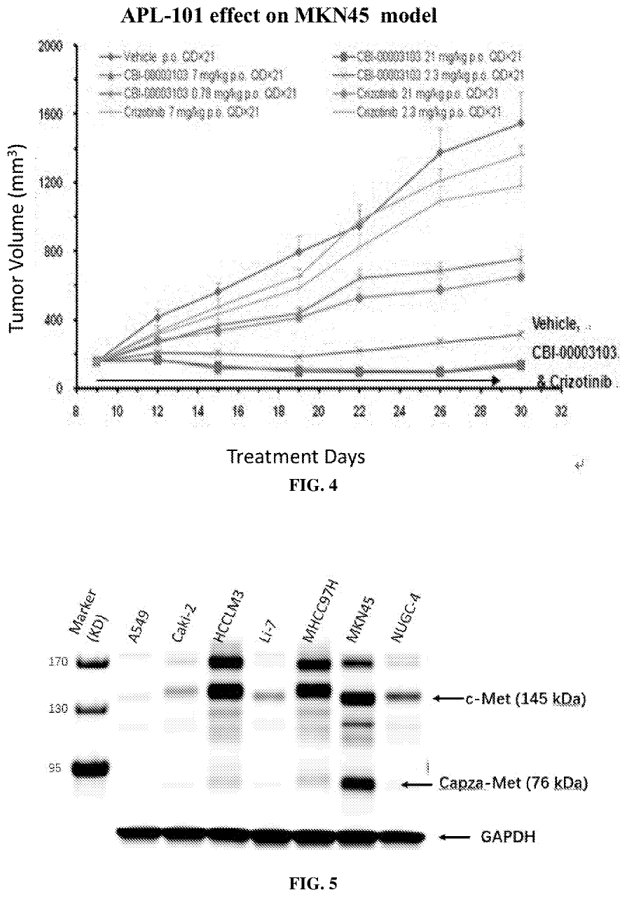

The inventors tested 3 PDX tumors and one CDX (cell line derived xenograft) tumor (MKN45) harboring both fusions and amplifications of c-Met in vivo for sensitivity towards APL-101. As shown in FIGS. 1-4, all four tumor models showed exquisite sensitivity towards the c-Met inhibitor.

The results indicate that c-Met point mutations and fusions alone may not be sufficient to dictate sensitivity towards c-Met inhibitors, while point mutations and fusions and amplification and high levels of expression (at transcription and protein level) together may. Along with recent findings in the clinic that almost all c-Met exon 14 skipping patients whose c-Met expression levels are low, do not respond to c-Met inhibitors, whereas those with high expression of c-Met with exon 14 skipping shows sensitivity towards inhibitor treatment, a common theme in c-Met is emerging that c-Met genetic mutation may require more than one successive event to permit sensitivity towards c-Met inhibitors. This may have significant implications in designing clinical studies to direct the therapies to the patients with the best chance of obtaining clinical benefit.

TABLE 1
The amino acid change of c-Met protein
caused by c-MET gene mutation

| No. | Amino_acid_change |
|---|---|
| 1 | p.K6N |
| 2 | p.V13L |
| 3 | p.G24E |
| 4 | p.E34A |
| 5 | p.E34K |
| 6 | p.A347T |
| 7 | p.M35V |
| 8 | p.A48G |
| 9 | p.H60Y |
| 10 | p.D94Y |
| 11 | p.G109R |
| 12 | p.S135N |
| 13 | p.D153A |
| 14 | p.H159R |
| 15 | p.E167K |
| 16 | p.E168D |
| 17 | p.E168K |
| 18 | p.T17I |
| 19 | p.P173A |
| 20 | p.R191W |
| 21 | p.S197F |
| 22 | p.T200A |
| 23 | p.A204PfsTer3 |
| 24 | p.F206S |
| 25 | p.L211W |
| 26 | p.G212V |
| 27 | p.S213L |
| 28 | p.L213F |
| 29 | p.T222M |
| 30 | p.L238YfsTer25 |
| 31 | p.S244Y |
| 32 | p.I259F |
| 33 | p.T273N |
| 34 | p.F281L |
| 35 | p.E293K |
| 36 | p.K305_R307del |
| 37 | p.A320V |
| 38 | p.S323G |
| 39 | p.G344R |
| 40 | p.M362T |
| 41 | p.P366S |
| 42 | p.N375K |
| 43 | p.N375S |
| 44 | p.V378I |
| 45 | p.H396Q |
| 46 | p.C397S |
| 47 | p.S406Ter |
| 48 | p.F430L |
| 49 | p.F445L |
| 50 | p.L455I |
| 51 | p.T457HfsTer21 |
| 52 | p.P472S |
| 53 | p.E493K |
| 54 | p.Y501H |
| 55 | p.L515M |
| 56 | p.L530V |
| 57 | p.V546M |
| 58 | p.R547Q |
| 59 | p.S572N |
| 60 | p.R591W |
| 61 | p.K595T |
| 62 | p.R602K |
| 63 | p.L604I |
| 64 | p.L604V |
| 65 | p.T618M |
| 66 | p.T621I |
| 67 | p.M630T |
| 68 | p.M636V |
| 69 | p.I638L |
| 70 | p.G645R |
| 71 | p.T646A |
| 72 | p.T651S |
| 73 | p.G679V |
| 74 | p.R731Q |
| 75 | p.S752Y |
| 76 | p.F753C |
| 77 | p.P761S |
| 78 | p.V765D |
| 79 | p.K783E |
| 80 | p.F804C |
| 81 | p.R811H |
| 82 | p.E815D |
| 83 | p.T835PfsTer7 |
| 84 | p.G843R |
| 85 | p.I852F |
| 86 | p.I852N |
| 87 | p.Y853H |
| 88 | p.D882N |
| 89 | p.D882Y |
| 90 | p.E891K |
| 91 | p.L905_H906delinsY |
| 92 | p.H906Y |
| 93 | p.V910F |
| 94 | p.Q931R |
| 95 | p.V937I |
| 96 | p.V941L |
| 97 | p.Q944Ter |
| 98 | p.L967F |
| 99 | p.R976T |
| 100 | p.L982_D1028del |
| 101 | p.R988C |
| 102 | p.Y989C |
| 103 | p.Y989Ter |
| 104 | p.A991P |
| 105 | p.T995N |
| 106 | p.V1007I |
| 107 | p.P1009S |
| 108 | p.T1010I |
| 109 | p.M1013I |
| 110 | p.S1015Ter |
| 111 | p.D1028H |
| 112 | p.S1033L |
| 113 | p.R1040Q |
| 114 | p.Y1044C |
| 115 | p.Q1085K |
| 116 | p.G1120V |
| 117 | p.G1137A |
| 118 | p.L1158F |
| 119 | p.S1159L |
| 120 | p.R1166Q |
| 121 | p.R1166Ter |
| 122 | p.R1184Q |
| 123 | p.R1188Ter |
| 124 | p.D1198H |
| 125 | p.V1238I |
| 126 | p.A1239V |
| 127 | p.D1240N |
| 128 | p.Y1248H |
| 129 | p.A1299V |
| 130 | p.L1330YfsTer4 |
| 131 | p.I316M |
| 132 | p.I333L |
| 133 | p.A1357V |
| 134 | p.V1368D |
| 135 | p.A1381T |
| 136 | p.L1386V |
| 137 | p.S1403Y |

TABLE 2
The partner genes involved in c-MET gene fusions
identified in tumor cell lines and PDX models

| Up gene | Dw gene |
|---|---|
| ACTG1 | MET |
| ANXA2 | MET |
| CAPZA2 | MET |
| DNAL1 | MET |
| FN1 | MET |
| GTF2I | MET |
| KANK1 | MET |
| MECP2 | MET |
| MET | AGMO |
| MET | ANXA2 |
| MET | CAPZA2 |
| MET | CAV1 |
| MET | IGF2 |
| MET | INTU |
| MET | ITGA3 |
| MET | NEDD4L |
| MET | PIEZO1 |
| MET | PLEC |
| MET | POLR2A |
| MET | SLC16A3 |
| MET | SMYD3 |
| MET | ST7 |
| MET | STEAP2-AS1 |
| MET | TES |
| MET | TTC28-AS1 |
| MGEA5 | MET |
| PPM1G | MET |
| RPS27A | MET |
| ST7 | MET |
| TES | MET |
| ZKSCAN1 | MET |

TABLE 3
In vitro analysis of APL-101 in c-Met point mutation cell lines

| | | | | | | MET amplification | |
|---|---|---|---|---|---|---|---|
| | | | Tumor | | IC50 | (copy number, | Max |
| No. | Mutation | Cell line | Type | Domain | (μM) | Microarray) | inhibition |
| 1 | p.K6N | OE19 | Esophagus | | >10 | 2.3746 | 29.90% |
| 2 | p. E34K | HCC1588 | Lung | | >10 | No data | 11.60% |
| 3 | | LS513 | Colon | | >10 | 2.6764 | 18.21% |
| 4 | p. E168D | SW1573 | Lung | SEMA domain | >10 | 1.7715 | 30.73% |
| 5 | | SU-DHL-10 | Lymphoma | | >10 | 2.8769 | 33.74% |
| 6 | p. I316M | P3HR-1 | Lymphoma | | >10 | 1.9243 | 24.08% |
| 7 | | PLC/PRF/5 | Liver | | >10 | 2.9905 | 11.49% |
| 8 | p. A347T | SU-DHL-5 | Lymphoma | | >10 | 2.0478 | 15.99% |
| 9 | p. M362T | SJSA-1 | Bone | | >10 | 2.09 | 4.33% |
| 10 | p. N375S | HCC2218 | Breast | | >10 | 2.8288 | 29.42% |
| 11 | | NCI-H209 | Lung | | >10 | 2.8073 | 10.62% |
| 12 | p. R988C | H69AR | Lung | Juxta-membrane | >10 | No data | 6.75% |
| 13 | | NCI-H1437 | Lung | Domain | >10 | 2.3229 | 13.22% |
| 14 | p. T1010I | HCC1428 | Breast | | >10 | 2.3243 | 10.40% |
| 15 | | HT-1376 | Bladder | | >10 | 2.049 | 28.72% |
| 16 | p. V1238I | Caki-1 | Kidney | TK domain | >10 | 2.9118 | 28.98% |
| 17 | p. A1239V | A2780 | Ovary | | >10 | 2.0124 | 34.05% |
| 18 | p. V1368D | HEC-1-A | Uterus | | >10 | 1.9443 | 23.64% |
| 19 | Exon 14 | HS746.T | Gastric | | 0.011 | 12.8616 | 62.46% |
| | skipping | | | | | | |

TABLE 4
Fusion genes involving c-MET in various Human tumor cell lines

| | | | | Up | | Dw | | | | | | |
|---|---|---|---|---|---|---|---|---|---|---|---|---|
| | Up- | Down- | | fusion | | fusion | If | | | | | |
| | stream | stream | | paint | | point | validated | If | Span | June | Span | June |
| Cell | gene | gene | Up | genome | Dw | genome | in current | in- | num by | num by | num by | num by |
| Line | (Up) | (Dw) | chr | position | chr | position | assay | frame | Soapfuse* | Soapfuse* | defuse^{†} | defuse^{†} |
| Caki-2 | CAPZA2 | MET | chr7 | 116501404 | chr7 | 116335804 | NO | NO | undetected | undetected | 14 | 7 |
| Caku-2 | CAPZA2 | MET | chr7 | 116502704 | chr7 | 116422042 | Yes | Yes | 9 | 2 | 14 | 10 |
| Caki-2 | CAPZA2 | MET | chr7 | 116502704 | chr7 | 116435709 | Yes | Yes | undetected | undetected | 14 | 2 |
| Caki-2 | MET | CAPZA2 | chr7 | 116437021 | chr7 | 116561157 | NO | NO | undetected | undetected | 14 | 2 |
| HCCLM3 | ANXA2 | MET | chr15 | 60686773 | chr7 | 116335804 | NO | NO | undetected | undetected | 2 | 4 |
| HCCLM3 | MECP2 | MET | chrX | 153313633 | chr7 | 116335804 | Yes | NO | undetected | undetected | 2 | 3 |
| HCCLM3 | MET | CAV1 | chr7 | 116312631 | chr7 | 116199000 | Yes | NO | undetected | undetected | 13 | 2 |
| HCCLM3 | MET | CAV1 | chr7 | 116340338 | chr7 | 116199000 | Yes | NO | 6 | 18 | 13 | 12 |
| HCCLM3 | MET | POLR2A | chr7 | 116438207 | chr7 | 7416908 | NO | NO | undetected | undetected | 3 | 2 |
| HCCLM3 | RPS27A | MET | chr2 | 55462700 | chr7 | 116435808 | NO | NO | 5 | 3 | undetected | undetected |
| HCCLM3 | RPS27A | MET | chr2 | 55462719 | chr7 | 116435709 | NO | NO | 5 | 9 | undetected | undetected |
| Li-7 | CAPZA2 | MET | chr7 | 116501404 | chr7 | 116335804 | NO | NO | undetected | undetected | 1 | 3 |
| Li-7 | CAPZA2 | MET | chr7 | 116538889 | chr7 | 116403104 | NO | Yes | undetected | undetected | 1 | 2 |
| MHCC97-H | ANXA2 | MET | chr15 | 60686773 | chr7 | 116335804 | NO | NO | undetected | undetected | 2 | 4 |
| MHCC97-H | ZKSCAN1 | MET | chr7 | 99616972 | chr7 | 116335804 | NO | NO | undetected | undetected | 1 | 5 |
| MKN45 | CAPZA2 | MET | chr7 | 116501404 | chr7 | 116335804 | NO | NO | undetected | undetected | 8 | 10 |
| MKK45 | CAPZA2 | MET | chr7 | 116538889 | chr7 | 116403104 | Yes | Yes | 2 | 13 | 8 | 11 |
| NUGC-4 | ZKSCAN1 | MET | chr7 | 99616972 | chr7 | 116335804 | NO | NO | undetected | undetected | 2 | 3 |

*&^{†}Different software used for gene fusion prediction. Indicate the reads number of the gene fusion found.

TABLE 5
The transcripts derived from fusion genes involving c-MET in diffrent cell lines

| | | | Exons | If |
|---|---|---|---|---|
| | | | of MET | encoding |
| | | | pre- | a pro- |
| | | | sented | ductive |
| Cell | | | in the | kinase |
| line | Fusion | | fusion | domain |
| Name | point | Sanger sequence result | gene | from MET |
| Caki-2 | CAPZA2:: | GTTTGTCCACAGAGACTTGGCTGCAAGAAACTGTATGGGAAGATGGCGGATCTGGAGG | 18-21 | No |
| | MET chr7 | AGCAGTTGTCTGATGAAGAGAAG\|AATCCAACTGTAAAAGATCTTATTGGCTTTGGTCTT | | |
| | (116502704- | CAAGTAGCCAAACCGANNAANTNTCTGCAAGCAAAAA (SEQ ID NO: 8) | | |
| | 116422042) | | | |
| Caki-2 | CAPZA2:: | TTGTCTGATGAGAGAAG\|TGGTCCTTTTGGCGTGCTCCTCTGGGAGCTGATGACAAGAGG | 20 [symbol] 21 | No |
| | MET chr7 | AGCCCCACCTTATCCTGATGTAAACACCTTTGATATAACTGTTTACTTGTTGCAAGGGAG | | |
| | (116502704- | AAGACTCCTACAACCCGAATACTGCCCAGACCCCTTATATGAAGTAATGCTAAAATGCT | | |
| | 116435709) | GGCACCCTAAAGCCGAAATGCGCCCATCCTTTTCTGATGTTTGTCGCCAGAAGGAAAGAT | | |
| | | GGCGGATCTGGAGGAGCAGTTGTCTG (SEQ ID NO: 9) | | |
| HCCLM3 | MECP2::MET | AAGAGTTTAGCAGAATGCTTCCCATATGATAAACCTCTGATAATGAAGGCCCCCGCTGT | Out of | No |
| | chrX::chr7 | GCTTGCACCTGGCATCCTCGTGCTCCTGTTTACCTTGGTGCAGAGGAGCAATGGGGAGT | frame | |
| | (153313633- | GTAAGCCTCCCAAGTAGCTGAGACTACAG\|GGTGGTGATGAAGAGTAAATCA | | |
| | 116335804) | (SEQ ID NO: 10) | | |
| HCCLM3 | MET::CAV1 | CTTCTCCACGGTTCCTGGGCACCGAAAG\|ATTGACTGAAGANGNGATGCAAACCAGAAG | 1 | No |
| | ch7 | GGACACACAGTTTTGACGGCATTTGGAAGGCC (SEQ ID NO: 11) | | |
| | (116312631- | | | |
| | 116199000) | | | |
| HCCLM3 | MET::CAV1 | TGTGTGCATTCCCTATCAAATATGTCAACGACTTCTTCAACAAGATCGTCAACAAAAC | 1 [symbol] 2 | NO |
| | chr7 | AATGTGAGATGTCTCCAGCTTTTTACGGACCCAATCATGAGCACTGCTTTAATAGG AT | | |
| | (116340338- | TGACTTTGAAGATGTGATTGCAGAACCAGAAGGGACACACAGTTTTGACGGCATTTGGA | | |
| | 116199000) | AGGCCAGCTTCACCACCTTCACTGTGACGAAATACTGGTTTTACCGCTTGCTGTCTGCCC | | |
| | | TCTTTGGCATCCCGATGGCACTCATCTGGGGCATTTACTTCGCCAATTGTTCGCACAAAG | | |
| | | CAAGCCAGATTCTGCCGAACCAATGGATCCATCTGCCA (SEQ ID NO: 12) | | |
| MKN45 | CAPZA2:: | TGCATTTGCACAGTATAACTTGGACCAGTTTACTCCAGTAAAAATTGAAGGTTATGAAG | 11-21 | Yes |
| | MET chr7 | ATCAG\|GCATGTCAACATCGCTCTAATTCAGAGATAATCTGTTGTACCACTCCTTCCCTG | | |
| | (116538889- | CAACAGCTGAATCTGCAACTCCCCTTCAATGATGTTCGGTTACTGCTTAATAATGACAA | | |
| | 116403104) | TCTTCTCAGGGAAGGAGCAGCCCA (SEQ ID NO: 13) | | |

[symbol]

indicates data missing or illegible when filed

TABLE 6
Models that harbor MET gene fusions

| Item | Cell line subset | PDX model subset | Sum |
|---|---|---|---|
| Number of models | 9 | 21 | 30 |
| with Met fusion(s) | | | |
| Total number of | 976 | 1611 | 2587 |
| models screened | | | |
| Percentage | 0.92% | 1.30% | 1.16% |

TABLE 7
Kinase live (exon 15-21 intact) fusions

| Item | Cell line subset | PDX model subset | Sum |
|---|---|---|---|
| Number of models | 9 | 21 | 30 |
| with Met fusion(s) | | | |
| Number of models | 7 | 14 | 21 |
| with kinase live | | | |
| fusion (exon1~14 | | | |
| fusion) | | | |
| Percentage | 77.78% | 66.67% | 70.00% |

TABLE 8
Fusion partners identified in cell lines and PDX models.

| No. | Gene | Partner | Breakpoint |
|---|---|---|---|
| 1 | MET | ACTG1 | Exon 15, 16 |
| 2 | MET | ANXA2 | Exon 2, 1 |
| 3 | MET | CAPZA2 | Exon 2, 6, 18, 20, 21 |
| 4 | MET | DNAL1 | Exon 14 |
| 5 | MET | FN1 | Exon 3 |
| 6 | MET | GTF2I | Exon 15, 16 |
| 7 | MET | KANK1 | Exon 15, 16 |
| 8 | MET | MECP2 | Exon 2 |
| 9 | MET | AGMO | Exon 1 |
| 10 | MET | CAV1 | Exon 2 |
| 11 | MET | INTU | Exon 1 |
| 12 | MET | ITGA3 | Exon 21 |
| 13 | MET | NEDD4L | Exon 10 |
| 14 | MET | PIEZO1 | Exon 1 |
| 15 | MET | PLEC | Exon 21 |
| 16 | MET | POLR2A | Exon 21 |
| 17 | MET | SLC16A3 | Exon 7, 21 |
| 18 | MET | SMYD3 | No data |
| 19 | MET | ST7 | Exon 2, 3 |
| 20 | MET | STEAP2-AS1 | Exon 1, 2 |
| 21 | MET | TES | Exon 1 |
| 22 | MET | TTC28-AS1 | Exon 1 |
| 23 | MET | MGEA5 | Exon 21 |
| 24 | MET | PPM1G | Exon 21 |
| 25 | MET | RPS27A | Exon 20 |
| 26 | MET | ZKSCAN1 | Exon 2 |

TABLE 9
c-Met fusions identified in cell lines and PDX models

| | Number of | Percentage of total |
|---|---|---|
| | models with | models with MET |
| Cancer Type | MET fusion(s) | fusion(s) |
| Cholangiocarcinoma | 3 | 10% |
| Colorectal Cancer | 1 | 3.33% |
| Esophageal Cancer | 2 | 6.67% |
| Gastric Cancer | 5 | 16.67% |
| Head and Neck Cancer | 1 | 3.33% |
| Liver Cancer | 5 | 16.67% |
| Lung Cancer | 10 | 33.33% |
| Metastatic Cancer | 1 | 3.33% |
| Kidney Cancer | 1 | 3.33% |
| Uterine Cancer | 1 | 3.33% |
| Total | 30 | 100% |

TABLE 10
Transcript levels of wild type c-MET and fusion genes
involving c-MET in different tumor cell lines

| | Transcript Level (Fold/GAPDH) | | | |
|---|---|---|---|---|
| Gene information | Caki-2 | MKN45 | HCCLM3 | MHCC97-H |
| GAPDH | 1.00 | 1.00 | 1.00 | 1.00 |
| Wild type c-MET | 105.71 | 315.27 | 757.86 | 7181.73 |
| CAPZA2-MET | 1.79 | Non-existing | Non-existing | Non-existing |
| (116502704-116422042) | | | | |
| CAPZA2-MET | 0.29 | Non-existing | Non-existing | Non-existing |
| (116502704-116435709) | | | | |
| CAPZA2-MET | Non-existing | 6.19 | Non-existing | Non-existing |
| (116538889-116403104)) | | | | |
| MET-CAV1 | Non-existing | Non-existing | 0.67 | Non-existing |
| (116340338-116199000) | | | | |

TABLE 11
In vitro analysis of APL-101 in c-Met fusion cell lines.

| | | | | | | Met amplification | |
|---|---|---|---|---|---|---|---|
| Cell | Cell | | | | | (copy number, | Max |
| No. | Line | Up gene | Dw gene | Breakpoint | IC50(uM) | microarray) | inhibition |
| 1 | Caki-2 | CAPZA2 | MET | Exon2, 18, | >10 uM | 2.0478 | −12.03% |
| | | | | 20, 21 | | | |
| 2 | HCCLM3 | ANXA2 | MET | Exon2 | 0.061 | overexpression | 67.13% |
| | | MECP2 | MET | Exon2 | | Likely amp | |
| | | MET | CAV1 | Exon2 | | | |
| | | MET | POLR2A | Exon21 | | | |
| | | RPS27A | MET | Exon20 | | | |
| 3 | Li-7 | CAPZA2 | MET | Exon2 | >10 uM | 2.3256 | |
| 4 | MHCC97-H | ANXA2 | MET | Exon2 | 0.024 | overexpression | 76.19% |
| | | ZKSCAN1 | MET | Exon2 | | Likely amp | |
| 5 | MKN45 | CAPZA2 | MET | Exon2, Exon11 | 0.018 | 12.3634 | 87.27% |
| 6 | NUGC-4 | ZKSCAN1 | MET | Exon2 | >10 uM | | 27.98% |

1. A method for predicting responsiveness of a subject having cancer to treatment with a c-Met inhibitor, said method comprising
detecting an expression level of active c-Met in a cancer sample from a subject;
detecting a c-Met gene mutation, a c-Met gene fusion or a c-Met gene amplification in the cancer sample;
determining that the expression level of active c-Met is higher than a reference expression level of c-Met; and
determining that the subject is likely to respond to treatment with the c-Met inhibitor.

2. The method of claim 1, wherein the expression level of active c-Met is a nRNA level or a protein level.

3. The method of claim 1, wherein the active c-Met is a wild-type c-Met, a mutated c-Met, a c-Met fusion or a combination thereof.

4. The method of claim 1, wherein the c-Met gene mutation results in a mutated c-Met protein with an amino acid change selected from the group consisting of K6N, V13L, G24E, E34A, E34K, A347T, 1\435V, A48G, HWY, D94Y, GIO9R, S135N, D153A, H159R, E167K, E168D, E168K, T171, P173A, R.191W, S197F, T200A, A204PfsTer3, F206S, L211W, G212V, S213L, T222M, L238YfsTer25, S244Y, I259F, T273N, F281L, E293K, K305_R307del, A320V, S323G, G344R, M362T, N375K, N375S, V378I, H396Q, C397S, S406Ter, F430L, F445L, L455I, T457HfsTer21, P472S, E493K, Y501H, L515M, L530V, V546M, R547Q, S572N, R591W, K595T, R602K, L6041, L604V, T618M, T621I, M630T, M636V, I638L, G645R, T646A, T651S, G679V, R731Q, S752Y, F753C, P761S, V765D, K783E, F804C, R811H, E815D, T835PfsTer7, G843R, I852F, I852N, Y853H, D882N, D882Y, E891K, L905_H906delinsY, H906Y, V910F, Q931R, V937I, V941L, Q944Ter, L967F, R976T, L982_D1028del, R988C, Y989C, Y989Ter, A991P, T995N, V1007I, P1009S, T1010I, M1013I, S1015Ter,D1028H, S1033L, R1040Q, Y1044C, Q1085K, G1120V, G113 7A, L1158F, S1159L, R1166Q, R1166Ter, R1184Q, R1188Ter, D1198H, V1238I, A1239V, D1240N, Y1248H, A1299V, L1330YfsTer4, I316M, I333L, A1357V, V1368D, A1381T, L1386V and S1403Y and a combination thereof.

5. The method of claim 1, wherein the c-Met gene fusion results in a gene fusion product selected from the group consisting of ACTG1/MET, ANXA2/MET, CAPZA2/MET, DNAL1/MET, FN1/MET, GTF2I/MET, KANK1/MET, MECP2/MET, MET/AGMO, MET/ANXA2, MET/CAPZA2, MET/CAV1, MET/IGF2, MET/INTU, MET/ITGA3, MET/NEDD4L, MET/PIEZO1, MET/PLEC, MET/POLR2A, MET/SLC16A3, MET/SMYD3, MET/ST7, MET/STEAP2-AS1, MET/TES, MET/TTC28-AS1, MGEA5/MET, PPM1G/MET, RPS27A/MET, ST7/MET, TES/MET, ZKSCAN1/MET and a combination thereof.

6. The method of claim 1, wherein the cancer is selected from the groups consisting of lung cancer, melanoma, renal cancer, liver cancer, myeloma, prostate cancer, breast cancer, colorectal cancer, pancreatic cancer, thyroid cancer, hematological cancer, leukemia and non-Hodgkin's lymphoma.

7. The method of claim 1, wherein the cancer is non-small cell lung cancer (NSCLC), renal cell carcinoma or hepatocellular carcinoma.

8. The method of claim 1, wherein the cancer sample is tissue or blood.

9. The method of claim 1, wherein the c-Met gene mutation, the c-Met gene fusion, or the c-Met gene amplification is detected using next generation sequencing.

10. The method of claim 1, wherein the expression level of active c-Met is detected using an amplification assay, a hybridization assay, a sequencing assay, or an immunoassay.

11. The method of claim 1, wherein the c-Met inhibitor is selected from the group consisting of Crizotinib, Cabozantinib, Tepotinib, AMG337, APL-101 (PLB1001, bozitinib), SU11274, PHA665752, K252a, PF-2341066, AM7, KNJ-38877605, PF-04217903, MK2461, GSK1363089 (XL880, foretinib), AMG458, Tivantinib (ARQ197), INCB28060 (INC280, capmatinib), E7050, BMS-777607, savolitinib (volitinib), HQP-8361, merestinib, ARGX-111, onartuzumab, rilotumumab, emibetuzumab, and XL184.

12. The method of claim 1, wherein the c-Met inhibitor is an anti-c-Met antibody.

13. The method of claim 1, wherein the c-Met inhibitor comprises a compound of the following formula 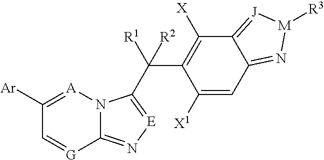
wherein:
R¹ and R² are independently hydrogen or halogen;
X and X′ are independently hydrogen or halogen;
A and G are independently CH or N, or CH═G is replaced with a sulfur atom;
E is N;
J is CH, S or NH;
M is N or C;
Ar is aryl or heteroaryl, optionally substituted with 1-3 substituents independent selected from: C₁₋₆alkyl, C₁₋₆alkoxyl halo C₁₋₆alkyl, halo C₁₋₆ alkoxy, C₃₋₇cycloalkyl, halogen, cyano, amino, —CONR⁴R⁵, —NHCOR⁶, —SO₂NR⁷R⁸, C₁₋₆alkoxyl, C₁₋₆alkyl-, amino C₁₋₆alkyl heterocyclyl and heterocyclyl-C₁₋₆alkyl-, or two connected substituents together with the atoms to which they are attached form a 4-6 membered lactam fused with the aryl or heteroaryl;
R3 is hydrogen, C₁₋₆alkyl, C₁₋₆alkoxyl, haloC₁₋₆alkyl, halogen, amino, or —CONH-C₁₋₆alkyl- heterocyclyl;
R⁴ and R5 are independently hydrogen, C₁₋₆alkyl, C₃₋₇cycloalkyl, heterocyclyl-C₁₋₆alkyl, or R⁴ and R⁵ together with the N to which they are attaches form a heterocyclyl;
R⁶ is C₁₋₆alkyl or C₃₋₇cycloalkyl; and
R⁷ and R⁸ are independently hydrogen or C₁₋₆-alkyl.

14. A method for treating a subject having cancer, the method comprising:
detecting an expression level of active c-Met in a cancer sample from a subject;
detecting a c-Met gene mutation, a c-Met gene fusion or a c-Met gene amplification in the cancer sample;
determining that the expression level of active c-Met is higher than a reference expression level of c-Met;
determining that the subject is likely to respond to treatment with a c-Met inhibitor; and
administering to the subject the c-Met inhibitor.

15-18. (canceled)

19. A method for treating a subject having cancer, the method comprising:
administering to the subject a therapeutically effective amount of a c-Met inhibitor, wherein a cancer sample from the subject has been determined to comprise:
(i) an increased expression level of active c-Met compared to a reference expression level of c-Met; and
(ii) a c-Met gene mutation, a c-Met gene fusion or a c-Met gene amplification.

20. The method of claim 19, wherein the expression level of active c-Met is a mRNA level or a protein level.

21. The method of claim 19. wherein the c-Met gene mutation results in a mutated c-Met protein with an amino acid change selected from the group consisting of K6N, V13L, G24E, E34A, E34K, A347T, M35V, A48G, H60Y, D94Y, G109R, S135N, D153A, H159R, E167K, E168D, E168K, T17I, P173A, R191W, S197F, T200A, A204PfsTer3, F206S, L211W, G212V, S213L, T222M, L238YfsTer25, S244Y, I259F, T273N, F281L, E293K, K305_R307del, A320V, S323G, G344R, M362T, N375K, N375S, V378I, H396Q, C397S, S406Ter, F430L, F445L, L455I, T457HfsTer21, P472S, E493K, Y501H, L515M, L530V, V546M, R547Q, S572N, R591W, K595T, R602K, L604I, L604V, T618M, T621I, M630T, M636V, I638L, G645R, T646A, T651S, G679V, R731Q, S752Y, F753C, P761 S, V765D, K783E, F804C, R811H, E815D, T835PfsTer7, G843R, I852F, I852N, Y853H, D882N, D882Y, E891K, L905_H906delinsY, H906Y, V910F, Q931R, V937I, V941L, Q944Ter, L967F, R976T, L982_D1028del, R988C, Y989C, Y989Ter, A991P, T995N, V1007I, P1009S, T1010I, M1013I, S1015Ter, D1028H, S1033L, R1040Q, Y1044C, Q1085K, G1120V, G-1137A, L1158F, S1159L, R1166Q, R1166Ter, R1184Q, R1188Ter, D1198H, V1238I, A1239V, D1240N, Y1248H, A1299V, L1330YfsTer4, I316M, I333L, A1357V, V1368D, A1381T, L1386V and S1403Y and a combination thereof.

22. The method of claim 19, wherein the c-Met gene fusion results in a gene fusion product selected from the group consisting of ACTG1/MET, ANXA2/MET, CAPZA2/MET, DNAL1/MET, FN1/MET, GTF2I/MET, KANK1/MET, MECP2/MET, MET/AGMO, MET/ANXA2, MET/CAPZA2, MET/CAV1, MET/IGF2, MET/INTU, MET/ITGA3, MET/NEDD4L, MET/PIEZO, MET/PLEC, MFT/POLR2A, MET/SLC16A3, MET/SMYD3, MET/ST7, MET/STEAP2-AS1, MET/TES, MET/TTC28-AS1, MGEA5/MET, PPM1G/MET, RPS27A/MET, ST7/MET, TES/MET, ZKSCAN1/MET and a combination thereof.

23. The method of claim 19, wherein the cancer is selected from the groups consisting of lung cancer, melanoma, renal cancer, liver cancer, myeloma, prostate cancer, breast cancer, colorectal cancer, pancreatic cancer, thyroid cancer, hematological cancer, leukemia and non-Hodgkin's lymphoma.

24. The method of claim 19, wherein the c-Met inhibitor is selected from the group consisting of Crizotinib, Cabozantinib, Tepotinib, AMG337, APL-101 (PLB1001, bozitinib), SU11274, PHA665752, K252a, PF-2341066, AM7, JNJ-38877605, PF-04217903, MK2461, GSK1363089 (XL880, foretinib), AMG458, Tivantinib (ARQ197), INCB28060 (INC280, capmatinib), E7050, BMS-777607, savolitinib (volitinib), HQP-8361, merestinib, ARGX-111, onartuzumab, rilotuniumab, emibetuzumab, and XL184.
